SEQUENCE LISTING (15 sequences)

SEQ ID NO: 1 - protein, 107 aa, Artificial Sequence
  source: /note=“Description of Artificial Sequence Synthetic polypeptide”
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 2 - protein, 122 aa, Artificial Sequence [sequence omitted]

SEQ ID NO: 3 - protein, 107 aa, Artificial Sequence
  source: /note=“Description of Artificial Sequence Synthetic polypeptide”
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKR

SEQ ID NO: 4 - protein, 122 aa, Artificial Sequence
  source: /note=“Description of Artificial Sequence Synthetic polypeptide”
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSS

SEQ ID NO: 5 - protein, 122 aa, Artificial Sequence
  source: /note=“Description of Artificial Sequence Synthetic polypeptide”
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSS

SEQ ID NO: 6 - protein, 213 aa, Artificial Sequence
  source: /note=“Description of Artificial Sequence Synthetic polypeptide”
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 452 aa, Artificial Sequence
  source: /note=“Description of Artificial Sequence Synthetic polypeptide”
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 452 aa, Artificial Sequence
  source: /note=“Description of Artificial Sequence Synthetic polypeptide”
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 213 aa, Artificial Sequence
  source: /note=“Description of Artificial Sequence Synthetic polypeptide”
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 452 aa, Artificial Sequence
  source: /note=“Description of Artificial Sequence Synthetic polypeptide”
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 452 aa, Artificial Sequence
  source: /note=“Description of Artificial Sequence Synthetic polypeptide”
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCAVSNKALPAPIEATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 452 aa, Artificial Sequence
  source: /note=“Description of Artificial Sequence Synthetic polypeptide”
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 452 aa, Artificial Sequence
  source: /note=“Description of Artificial Sequence Synthetic polypeptide”
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 14 - protein, 452 aa, Artificial Sequence
  source: /note=“Description of Artificial Sequence Synthetic polypeptide”
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 452 aa, Artificial Sequence
  source: /note=“Description of Artificial Sequence Synthetic polypeptide”
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2011/0020322 A1
(43) Pub. Date: Jan. 27, 2011

(54) CRYSTALLIZATION OF ANTI-CD20 ANTIBODIES

(75) Inventors: James A. Wilkins, San Francisco, CA (US); Shadia Abike Oshodi, New York, NY (US); Brian Lobo, Carlsbad, CA (US); Timothy Breece, San Francisco, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 12/818,017

(22) Filed: Jun. 17, 2010

Related U.S. Application Data
(63) Continuation of application No. PCT/US2008/087008, filed on Dec. 16, 2008.
(60) Provisional application No. 61/016,288, filed on Dec. 21, 2007.

(51) Int. Cl. A61K 39/395; C07K 16/00

(52) U.S. Cl. 424/130.1; 530/387.1

(57) ABSTRACT

The present invention relates generally to crystalline forms of anti-CD20 antibodies and purification of anti-CD20 antibodies involving crystallization.

### FIELD OF THE INVENTION

The present invention relates generally to crystalline forms of anti-CD20 antibodies and purification of anti-CD20 antibodies involving crystallization.

### BACKGROUND OF THE INVENTION

### CD20 Antibodies

Rituximab (RITUXAN®) is a genetically engineered chimeric murine/human monoclonal antibody directed against the CD20 antigen. Rituximab is the antibody called “C2B8” in U.S. Pat. No. 5,736,137 issued Apr. 7, 1998 (Anderson et al.). Rituximab is indicated for the treatment of patients with relapsed or refractory low-grade or follicular, CD20-positive, B cell non-Hodgkin's lymphoma. In vitro mechanism of action studies have demonstrated that rituximab binds human complement and lyses lymphoid B cell lines through complement-dependent cytotoxicity (CDC) (Reff. et. al., Blood 83(2):435-445 (1994)). Additionally, it has significant activity in assays for antibody-dependent cellular cytotoxicity (ADCC). More recently, rituximab has been shown to have anti-proliferative effects in tritiated thymidine incorporation assays and to induce apoptosis directly, while other anti-CD19 and CD20 antibodies do not (Maloney et al., Blood 88(10):637a (1996)). Synergy between rituximab and chemotherapies and toxins has also been observed experimentally. In particular, rituximab. sensitizes drug-resistant human B cell lymphoma cell lines to the cytotoxic effects of doxorubicin, CDDP, VP-1 6, diphtheria toxin and ricin (Demidem et al., Cancer Chemotherapy & Radiopharmaceuticals 12(3):177-186 (1997)). In vivo preclinical studies have shown that rituximab depletes B cells from the peripheral blood, lymph nodes, and hone marrow of cynomolgus monkeys, presumably through complement and cell-mediated processes (Reff et al., Blood 83(2):435-445 (1994)).

2H7 (ocrelizumab) is a second generation humanized monoclonal antibody directed against the CD20 surface antigen on human B-cells. Ocrelizumab is currently tested in Phase III clinical trials for the treatment of rheumatoid arthritis (RA).

Patents and patent publications concerning CD20 antibodies include U.S. Pat. Nos. 5,776,456, 5,736,137, 6,399,061, and 5,843,439, as well as U.S. patent application Nos. US 2002/0197255A1, US 2003/0021781A1, US 2003/0082172 A1, US 2003/0095963 A1, US 2003/0147885 A1 (Anderson et al.); U.S. Pat. No. 6,455,043B1 and WO00/09160 (Grillo-Lopez, A.); WO00/27428 (Grillo-Lopez and White); WO00/27433 (Grillo-Lopez and Leonard); WO00/44788 (Braslawsky et al.); WO01/10462 (Rastetter, W.); WO01/10461 (Rastetter and White); WO01/10460 (White and Grillo-Lopez); U.S. application No. US2002/0006404 and WO02/04021 (Hanna and Hariharan); U.S. application No. US2002/0012665 A1 and WO01/74388 (Hanna, N.); U.S. application No. US 2002/0058029 A1 (Hanna, N.); U.S. application No. US 2003/0103971 A1 (Hariharan and Hanna); U.S. application No. US2002/0009444A1, and WO01/80884 (Grillo-Lopez, A.); WO01/97858 (White, C.); U.S. application No. US2002/0128488A1 and WO02/34790 (Reff, M.); W)02/060955 (Braslawsky et al.); WO2/096948 (Braslawsky et al.); WO02/079255 (Reff and Davies); U.S. Pat. No. 6,171,586B1, and WO98/56418 (Lam et al.); WO98/58964 (Raju, S.); WO99/22764 (Raju, S.); WO99/51642, U.S. Pat. No. 6,194,551B1, U.S. Pat. No. 6,242,195B1, U.S. Pat. No. 6,528,624B1 and U.S. Pat. No. 6,538,124 (Idusogie et al.); WO00/42072 (Presta, L.); WO00/67796 (Curd et al.); WO01/03734 (Grillo-Lopez et al.); U.S. application No. US 2002/0004587A1 and WO01/77342 (Miller and Presta); U.S. application No. US2002/0197256 (Grewal, I.); U.S. application No. US 2003/0157108 A1 (Presta, L.); U.S. Pat. Nos. 6,090,365B1, 6,287,537B1, 6,015,542, 5,843,398, and 5,595,721, (Kaminski et al.); U.S. Pat. Nos. 5,500,362, 5,677,180, 5,721,108, and 6,120,767 (Robinson et al.); U.S. Pat. No. 6,410,391B1 (Raubitschek et al.); U.S. Pat. No. 6,224,866B1 and WO00/20864 (Barbera-Guillem, B.); WO01/13945 (Barbera-Guillem, E.); WO00/67795 (Goldenberg); U.S. application No. US 2003/01339301 A1 and WO00/74718 (Goldenberg and Hansen); WO00/76542 (Golay et al.); WO01/72333 (Wolin and Rosenblatt); U.S. Pat. No. 6,368,596B1 (Ghetie et al.); U.S. application No. US2002/0041847 A1, (Goldenberg, D.); U.S. application No. US2003/0026801A1 (Weiner and Hartmann); WO02/102312 (Engleman, E.); U.S. patent application No. 2003/0068664 (Albitar et al.); WO03/002607 (Leung, S.); WO 03/049694 and US 2003/0185796 A1 (Wolin et al.); WO03/061694 (Sing and Siegall); US 2003/0219818 A1 (Bohen et al.); US 2003/0219433 A1 and WO 03/068821 (Hansen et al.), US 2006/0246004 (Adams et al.);U.S. Pat. No. 5,849,898 and EP application no, 330,191 (Seed et al.); U.S. Pat. No. 4,861,579 and EP332,865A2 (Meyer and Weiss); U.S. Pat. No. 4,861,579 (Meyer et al.) and WO95/03770 (Bhat et al.), each of which is expressly incorporated herein by reference,

Publications concerning therapy with Rituximab include: Perotta and Abuel “Response of chronic relapsing ITP of 10 years duration to Rituximab” Abstract #3360 Blood 10(1)(part 1-2): p. 88B (1998); Stashi et al., “Rituximab chimeric anti-CD20 monoclonal antibody treatment for adults with chronic idopathic thrombocytopenic purpura” Blood 98(4):952-957 (2001); Matthews, R. “Medical Heretics” New Scientist (7 Apr., 2001); Leandro et al., “Clinical outcome in 22 patients with rheumatoid arthritis treated with B lymphocyte depletion” Ann Rheum Dis 61:833-888 (2002); Leandro et al., “Lymphocyte depletion in rheumatoid arthritis: early evidence for safety, efficacy and dose response. Arthritis & Rheumatism 44(9): 5370 (2001); Leandro et al., “An open study of 13 lymphocyte depletion in systemic lupus erythematosus”, Arthritis & Rheumatism 46(1):2673-2677 (2002); Edwards and Cambridge “Sustained improvement in rheumatoid arthritis following a protocol designed to deplete B lymphocytes” Rheumatology 40:205-211 (2001); Edwards et al., “B-lymphocyte depletion therapy in rheumatoid arthritis and other autoimmune disorders” Biochem. Soc. Trans. 30(4):824-828 (2002); Edwards et al., “Efficacy and safety of Rituximab, a B-cell targeted chimeric monoclonal antibody: A randomized, placebo controlled trial in patients with rheumatoid arthritis. Arthritis & Rheumatism 46(9): S197 (2002); Levine and Pestronk “IgM antibody-related polyneuropathies: B-cell depletion chemotherapy using Rituximab” Neurology 52: 1701-1704 (1999); DeVita et al., “Efficacy of selective B cell blockade in the treatment of rheumatoid arthritis” Arthritis & Rheumatism 46:2029-2033 (2002); Hidashida et al., “Treatment of DMARD-Refractory rheumatoid arthritis with rituximab.” Presented at the Annual Scientific Meeting of the American College of Rheumatology; October 24-29; New Orleans. La. 2002; Tuscano, J. “Successful treatment of Infliximab-refractory rheumatoid arthritis with rituximab” Presented at the Annual Scientific Meeting of the American College of Rheumatology; October 24-29; New Orleans, La. 2002. Sarwal et al., N. Eng. J. Med. 349(2):125-138 (Jul. 10, 2003) reports molecular heterogeneity in acute renal allograft rejection identified by DNA microarray profiling.

Production of Antibodies in Mammalian Cell Cultures

Mammalian cells have become the dominant system for the production of mammalian proteins for clinical applications, primarily due to their ability to produce properly folded and assembled heterologous proteins, and their capacity for post-translational modifications. Chinese hamster ovary (CHO) cells, and cell lines obtained from various other mammalian sources, such as, for example, mouse myeloma (NS0), baby hamster kidney (BHK), human embryonic kidney (HEK-293) and human retinal cells have been approved by regulatory agencies for the production of biopharmaceutical products, including therapeutic antibodies. Of these, Chinese Hamster Ovary Cells (CHO) are among the most commonly used industrial hosts, which are commonly used for the production of heterologous proteins. Thus, methods for the large-scale production of antibodies in CHO, including dihydrofolate reductase negative (DHFR-) CHO cells, are well known in the art (see, e.g. Trill et al., Curr. Opin. Biotechnol. 6(5):553-60 (1995)).

Usually, to begin the production cycle, a small number of transformed recombinant host cells is allowed to grow in culture for several days. Once the cells have undergone several rounds of replication, they are transferred to a larger container where they are prepared to undergo fermentation. The media in which the cells are grown and the levels of oxygen. nitrogen and carbon dioxide that exist during the production cycle may have a significant impact on the production process. Growth parameters are determined specifically for each cell line and these parameters are measured frequently to assure optimal growth and production conditions.

When the cells grow to sufficient numbers, they are transferred to large-scale production tanks and grown for a longer period of time. At this point in the process, the recombinant protein can be harvested. Typically, the cells are engineered to secrete the polypeptide into the cell culture media, so the first step in the purification process is to separate the cells from the media. Harvesting usually includes centrifugation and filtration to produce a Harvested Cell Culture Fluid (HCCF). The media is then subjected to several additional purification steps that remove any cellular debris, unwanted proteins, salts, minerals or other undesirable elements. At the end of the purification process, the recombinant protein is highly pure and is suitable for human therapeutic use.

Although this process has been the subject of much study and improvements over the past several decades, the production of recombinant proteins, such as antibodies, is still not without difficulties. The purification steps are often time consuming, expensive, and introduce additional issues. With current improvements in antibody titers from manufacturing cell culture, purification of the antibody now requires chromatography columns of unwieldy sizes and large amounts of expensive chromatography resin. The use of Protein A affinity chromatography columns to remove CHO host cell proteins (CHOP) from CHO cell cultures is known to involve Protein A leaching, and requires a further purification step to remove the leached Protein A. In addition, large-scale production of polypeptides, such as antibodies, requires the use and handling of large volumes, which adds to the expense, and often makes it difficult to achieve satisfactory titers. Thus, there is a need for improved methods for the large-scale purification of recombinant polypeptides, such as antibodies. In view of their established therapeutic importance, it would be particularly desirable to provide an improved process for the purification of CD20 antibodies, that would allow the reduction of the number of purification process steps while maintaining comparable yields to traditional purification schemes using multiple chromatographic purification steps.

There have been limited reports on using crystallization as part of the purification process of heterologous proteins. U.S. Application Publication No. 2006/0009387 reports that the Apo2L/TRAIL protein shows a tendency for spontaneous crystallization under certain conditions, and, based on this finding, describes a method for the purification of Apo2L/TRAIL including a crystallization step.

### SUMMARY OF THE INVENTION

The present invention is based, at least in part, on the surprising finding that, although antibodies, especially full-length antibodies, are traditionally difficult to crystallize, CD20 antibodies can be successfully crystallized from Harvested Cell Culture Fluid (HCCF) of mammalian cell cultures. In particular, the invention includes the identification of conditions that allow the formation of CD20 antibody crystals, including large, uniform, CD20 antibody crystals, from HCCF. Accordingly, the present invention provides a process for purifying CD20 antibodies from mammalian cell cultures, including a crystallization step in the purification scheme. Incorporation of a crystallization step in the CD20 antibody purification scheme eliminates chromatographic steps and their inherent limits of scalability while maintaining comparable yields to traditional purification schemes that use multiple chromatographic purification steps, without crystallization. Accordingly, implementing crystallization into the purification process results in marked time and cost savings, without compromising efficiency, product yields or product quality.

In one aspect, the invention concerns a method of purifying a CD20 antibody from a mixture, comprising crystallizing the CD20 antibody and recovering the crystalline CD20 antibody from the mixture.

In another aspect, the invention concerns a method of purifying a CD20 antibody from a mixture comprising the steps of (a) crystallizing the CD20 antibody to yield CD20 antibody crystals, (b) dissolving the CD20 antibody crystals to obtain a CD20 antibody solution, (d) subjecting the CD20 antibody solution to purification on an anion exchange column, and (e) isolating the CD20 antibody.

The mixture can be any mixture comprising CD20 antibodies, such as any composition obtained during the recombinant production of CD20 antibodies from any eukaryotic or prokaryotic host cells.

In a particular embodiment, the mixture is a Harvested Cell Culture Fluid (HCCF) from mammalian cells, such as Chinese Hamster Ovary (CHO) cells; the HCCF may be concentrated beyond its original concentration out of the bioreactor.

In another embodiment, the purification is performed in the absence of a Protein A purification step.

In yet another embodiment, the purification is performed in the absence of a cation exchange chromatography step.

In a further embodiment, the purification is performed in the absence of both a Protein A purification step and a cation exchange purification step.

In another embodiment, the purification scheme comprises a viral filtration step and an anion exchange purification step, which are preferably employed subsequent to the to crystallization purification step.

In an additional embodiment, the purification method of the present invention consists essentially of or consists of the following steps: (a) crystallization of the CD20 antibody from concentrated HCCF, (b) dissolution of the CD20 crystals in a buffer, (c) passing the solution obtained through an anion exchange column, and (d) concentration of the eluate leaving the anion exchange column.

In all embodiments, the CD20 antibody can be any diagnostic or therapeutic CD20 antibody, including, without limitation, Rituximab (RITUXAN®), humanized anti-CD20 antibodies including humanized 2H7 and 2H7 variants, HuMaX-CD20 (Genmab), IMMU-106 (also known as veltuzumab or hA20; Immunomedics). Monoclonal antibodies are preferred, which may be chimeric, humanized or human. In all aspects, the term CD20 “antibody” or “CD20 binding antibody” specifically includes full length CD20 binding antibody, and antigen-binding fragments thereof such as Fab or F(ab′)₂. Thus, methods for the crystallization of CD20 binding antibodies and variants thereof are specifically included herein.

For example, the CD20 antibody may be selected from the group consisting of 2H7 CD20 antibody variants A-I listed in Table 1. In a particular embodiment, the CD20 antibody is selected from the group consisting of 2H7 CD20 antibody variants A, C and H listed in Table 1, having VL and VH pairs of SEQ ID NOs: 1 and 2; SEQ ID NOs: 3 and 4; and SEQ ID NOs: 3 and 5, respectively.

In a different aspect, the invention concerns a method of purifying a CD20 antibody from concentrated Harvested Cell Culture Fluid (HCCF) of mammalian cells, comprising the steps of (a) concentrating the HCCF, (b) diafiltering the HCCF with a high salt concentration at a pH that inhibits crystallization, (c) crystallizing the CD20 antibody by raising the pH, (d) dissolving the CD20 antibody crystals to obtain a CD20 antibody solution, (e) subjecting the CD20 antibody solution to purification on an anion exchange column, and (f) recovering the resultant purified CD20 antibody.

Just as before, exemplary CD20 antibodies may be selected from the group consisting of 2H7 CD20 antibody variants A-I listed in Table 1. In a particular embodiment, the CD20 antibody is selected from the group consisting of 2H7 CD20 antibody variants A, C and H listed in Table 1, having VL and VH pairs of SEQ ID NOs: 1 and 2; SEQ ID NOs: 3 and 4; and SEQ ID NOs: 3 and 5, respectively.

In another aspect, the methods of the present invention concern the crystallization and purification of antibody-like molecules comprising a CD20 binding sequence, such as CD20 binding immunoadhesins comprising a Fc region of an IgG. In one embodiment the CD20 binding immunoadhesin comprises the variable region of the humanized 2H7 antibody or one of its variants described in Table 1.

In all embodiments using HCCF as the starting mixture, the HCCF may be concentrated so that the CD20 antibody concentration is at a minimum of about 1.5 mg/ml. A CD20 antibody concentration of about 15 mg/ml provides good yield of antibody crystals with clearance of CHOP (CHO cell protein) but we have been able to obtain crystallization of the CD20 binding antibody at as low a concentration as 1.5 mg/ml.

In all embodiments, crystallization may be performed in a wide range of pH, such as, for example, at a pH of about 6.0 to about 8.0, or of 7.8+/−0.2.

Crystallization can be preformed in a wide concentration range, such as, for example at a temperature of about 4° C. to about 40° C., e.g. at a temperature of about 37° C.

Crystallization may be induced by one or more precipitants, such as one or more precipitants selected from the group consisting of PBS, NaCl, Na₂SO₄, KCl, K₂SO₄, Na₂HPO₄, and KH₂PO₄, in particular KH₂PO₄.

Crystallization is easier to achieve at higher protein concentrations but for practical reasons, the HCCF is not concentrated to a great extent.

In another aspect, the invention concerns a crystal of a CD20 antibody. The crystal may be present in different shapes, including, without limitation microneedle, needle, globular or globular peanut-shaped crystals, which can be present individually or in the form of various mixtures, in the presence or absence of an amorphous, non-crystalline precipitate.

In a further aspect, the invention concerns a composition comprising CD20 binding antibody crystals. The composition may, for example, be a pharmaceutical composition, comprising one or more pharmaceutically acceptable excipients.

The invention further concerns a method for treating a B cell malignancy or an autoimmune disease comprising administering to a mammalian subject an effective amount of a CD20 antibody purified by a method of the present invention. In specific embodiments, the autoimmune disease is selected from the group consisting of rheumatoid arthritis and juvenile rheumatoid arthritis, systemic lupus erythematosus (SLE) including lupus nephritis, Wegener's disease, inflammatory bowel disease, ulcerative colitis, idiopathic thrombocytopenic purpura (ITP), thrombotic thrombocytopenic purpura (TTP), autoimmune thrombocytopenia, multiple sclerosis, psoriasis, IgA nephropathy, IgM polyneuropathies, myasthenia gravis, ANCA associated vasculitis, diabetes mellitus, Reynaud's syndrome, Sjogren's syndrome, and Neuromyelitis Optica (NMO).

These and further embodiments will be apparent from the Examples provided below.

### BRIEF DESCRIPTION OF THE DRAWINGS

FIG. 1 is a microscopic view observation of a precipitate obtained from drug product containing 150 mg/ml 2H7 antibody dialyzed into beakers containing 20×PBS at 37° C., as described in Example 1.

FIG. 2 is a microscopic view of large “haystack” 2H7 antibody crystals grown from a solution containing 6 mg/ml 2H7 and 10×PBS at 24° C., as described in Example 2.

FIG. 3 is a microscopic view of needle-shaped and globular 2H7 antibody crystals grown from a solution containing 37.5 mg/ml 2H7 and 10×PBS at 37° C., as described in Example 2.

FIG. 4 is a microscopic view of thin needle 2H7 antibody crystals obtained grown from a solution containing 5 mg/ml 2H7 and 1 PBS at 4° C., as described in Example 2.

FIG. 5 is a microscopic view of large round ball-shaped and needle-shaped 2H7 antibody crystals grown from a solution containing 37.5 mg/ml 2H7 and 10×PBS at 37° C., as described in Example 3.

FIG. 6 is a microscopic view of thin. needle-shaped 2H7 antibody crystals obtained from a solution containing 5 mg/ml 2H7 and 10×PBS at 37° C., as described in Example 3.

FIG. 7 is a microscopic view of microneedles of 2H7 antibody crystals grown from a solution containing 75 mg/ml 2H7 and 300 mM Na₂HPO₄ at 37° C., as described in Example 3.

FIG. 8 is a microscopic view of large globular and peanut-shaped 2H7 antibody crystals obtained from a solution containing 17.5 mg/ml 2H7 and 500 mM KH₂PO₄ at 37° C., as described in Example 3.

FIG. 9 is a microscopic view of globular peanut shaped 2H7 antibody crystals grown from a solution containing 37.5 mg/ml 2H7 and 500 mM KH₂PO₄ at 37° C., as described in Example 3.

FIGS. 10A-H show microscopic views of 2H7 antibody crystals precipitated from a concentrated solution obtained from a conditioned pool of 2H7 that had been run through a Q-Sepharose chromatography step (hereinafter referred to as “Q-Pool”) containing 75 mg/ml, 37.5 mg/ml, 17.5 mg/ml or 5 mg/ml 2H7, using 10×PBS as a precipitant, in the presence (A, C, E, G) and absence (B, D, F, H) of Tween/Trehalose.

FIGS. 11A-C show microscopic views of 2H7 antibody crystals obtained from a Q-Pool containing 75 mg/ml, 37.5 mg/ml, or 17.5 mg/ml 2H7, using 1M KH₂PO₄ as a precipitant, in the presence (A, B, D) or absence (C, E) of Tween/Trehalose.

FIGS. 12A-B graphically summarize the effects of Trehalose and Tween on crystallization efficiency.

FIGS. 13A-H show microscopic views of 2H7 antibody crystals obtained from Harvested Cell Culture Fluid (HCCF) containing 15.5 mg/ml 2H7 in the presence of 10×PBS, 15×PBS, 500 mM KH₂PO₄, and 750 mM KH₂PO₄, respectively, using Q-Pool containing 15.5 mg/ml 2H7 as a control.

FIG. 14 is a graphical representation of the pH-dependence of crystallization efficiency from HCCF, using 500 mM KH₂PO₄ as the precipitant. The 2H7 concentration varied from 3 mg/ml to 15.5 mg/ml.

FIG. 15 shows HCCF dissolubility curves at 37° C., at one hour and 18 hours.

FIG. 16 shows HCCF dissolubility curves at 24° C., at one hour and 18 hours.

FIG. 17 shows HCCF dissolubility curves at 4° C., at one hour and 18 hours.

### DETAILED DESCRIPTION OF THE INVENTION

### A. Definitions

The term “antibody” is used in the broadest sense and specifically covers monoclonal antibodies (including full length or intact monoclonal antibodies), polyclonal antibodies, multivalent antibodies, multispecific antibodies (e.g., bispecific antibodies) formed from at least two intact antibodies, and antibody fragments (see below) so long as they exhibit the desired biological activity.

“Antibody fragments” comprise only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)₂ fragments, a bivalent fragment including two Fab′ fragments linked by a disulphide bridge at the hinge region; (ix) single chain antibody molecules (e.g. single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10):1057 1062 (1995); and U.S. Pat. No. 5,641,870).

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. Monoclonal antibodies are highly specific, being directed against a single antigen. In certain embodiments, a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations that typically include different antibodies directed against different determinants (epitopes), each monoclonal antibody is directed against a single determinant on the antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1991); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol, 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem, Soc. Transactions 23:1035-1038 (1995); Hurle and Gross, Curr. Op. Biotech. 5; 428-433 (1994); and U.S. Pat. Nos. 6,982,321 and 7,087,409. See also van Dijk and van de Winkel, Curr. Opin. Pharmacol, 5: 368-74 (2001). Human antibodies can be prepared by administering the antigen to a transgenic animal that has been modified to produce such antibodies in response to antigenic challenge, but whose endogenous loci have been disabled. e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA. 103:3557-3562 (2006) regarding human antibodies generated via a human B-cell hybridoma technology.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 14:309-314 (1996): Sheets et al. PNAS (USA) 95:6157-6162 (1998)); Hoogenboom and Winter, J. Mol. Biol., 227:381 (1991); Marks et al., J Mol. Biol., 222:581 (1991)). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p, 77 (1985); Boerner et al., J. Immunol., 147 (1):86-95 (1991); and U.S. Pat. No. 5,750,373.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody-dependent cellular toxicity.

The term “hypervariable region,” “HVR,” or “HV,” when used herein refers to the amino acid residues of an antibody which are responsible for antigen-binding. For example, the term hypervariable region refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., tamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact | |
|---|---|---|---|---|---|
| L1 | L24-L34 | L24-L34 | L26-L32 | L30-L36 | |
| L2 | L50-L56 | L50-L56 | L50-L52 | L46-L55 | |
| L3 | L89-L97 | L89-L97 | L91-L96 | L89-L96 | |
| H1 | H31-H35B | H26-H35B | H26-H32 | H30-H35B | (Kabat Numbering) |
| H1 | H31-H35 | H26-H35 | H26-H32 | H30-H35 | (Chothia Numbering) |
| H2 | H50-H65 | H50-H58 | H53-H55 | H47-H58 | |
| H3 | H95-H102 | H95-H102 | H96-H101 | H93-H101 | |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework Region” or “FR” residues are those variable domain residues other than the hypervariable region residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of 112 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

Throughout the present specification and claims, the Kabat numbering system is generally used when referring to a residue in the variable domain (approximately, residues 1-107 of the light chain and residues 1-113 of the heavy chain) (e.g, Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., Sequences of Proteins of Immunological Interest, 5th Ed. Public Health Service, National Institutes of Health, Bethesda, Md. (1991) expressly incorporated herein by reference). Unless stated otherwise herein, references to residues numbers in the variable domain of antibodies means residue numbering by the Kabat numbering system. Unless stated otherwise herein, references to residue numbers in the constant domain of antibodies means residue numbering by the EU numbering system (e.g., see U.S. Provisional Application No. 60/640,323, Figures for EU numbering).

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG₁ (including non-A and A allotypes), IgG₂, IgG₃, IgG₄, IgA₁, and IgA₂. The heavy chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The term “Fc region” is used to define the C-terminal region of an immunoglobulin heavy chain which may be generated by papain digestion of an intact antibody. The Fc region may be a native sequence Fc region or a variant Fc region. Although the boundaries of the Fc region of an immunoglobulin heavy chain might vary, the human IgG heavy chain Fc region is usually defined to stretch from an amino acid residue at about position Cys226, or from about position Pro230, to the carboxyl-terminus of the Fc region. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. The Fc region of an immunoglobulin generally comprises two constant domains, a CH2 domain and a CH3 domain, and optionally comprises a CH4 domain.

Unless indicated otherwise herein, the numbering of the residues in an immunoglobulin heavy chain is that of the HU index as in Kabat et al., supra. The “EU index as in Kabat” refers to the residue numbering of the human IgG1 EU antibody.

The “CH2 domain” of a human IgG Fc region (also referred to as “Cg2” domain) usually extends from an amino acid residue at about position 231 to an amino acid residue at about position 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22:161-206 (1985). The CH2 domain herein may be a native sequence CH2 domain or variant CH2 domain.

The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protroberance” in one chain thereof and a corresponding introduced “cavity” in the other chain thereof; see U.S. Pat. No. 5,821,333, expressly incorporated herein by reference). Such variant CH3 domains may be used to make multispecific (e.g. bispecific) antibodies as herein described.

“Hinge region” is generally defined as stretching from about Glu216, or about Cys226, to about Pro230 of human IgG1 (Burton, Molec. Immunol, 22:161-206 (1985)). Hinge regions of other IgG isotypes may be aligned with the IgG1 sequence by placing the first and last cysteine residues forming inter-heavy chain S—S bonds in the same positions. The hinge region herein may be a native sequence hinge region or a variant hinge region. The two polypeptide chains of a variant hinge region generally retain at least one cysteine residue per polypeptide chain, so that the two polypeptide chains of the variant hinge region can form a disulfide bond between the two chains. The preferred hinge region herein is a native sequence human hinge region, e.g. a native sequence human IgG1 hinge region.

A “functional Fc region” possesses at least one “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays known in the art for evaluating such antibody effector functions.

An “intact” antibody is one which comprises an antigen-binding variable region as well as a light chain constant domain (C_{L}) and heavy chain constant domains, C_{H}1, C_{H}2 and C_{H}3. The constant domains may be native sequence constant domains (e.g. human native sequence constant domains) or amino acid sequence variant thereof. Preferably, the intact antibody has one or more effector functions.

A “parent antibody” or “wild-type” antibody is an antibody comprising an amino acid sequence which lacks one or more amino acid sequence alterations compared to an antibody variant as herein disclosed. Thus, the parent antibody generally has at least one hypervariable region which differs in amino acid sequence from the amino acid sequence of the corresponding hypervariable region of an antibody variant as herein disclosed. The parent polypeptide may comprise a native sequence (i.e. a naturally occurring) antibody (including a naturally occurring allelic variant), or an antibody with pre-existing amino acid sequence modifications (such as insertions, deletions and/or other alterations) of a naturally occurring sequence. Throughout the disclosure, “wild type,” “WT,” “wt,” and “parent” or “parental” antibody are used interchangeably.

As used herein, “antibody variant” or “variant antibody” refers to an antibody which has an amino acid sequence which differs from the amino acid sequence of a parent antibody. In certain embodiments, the antibody variant will have an amino acid sequence from about 75% to less than 100% amino acid sequence identity or similarity with the amino acid sequence of either the heavy or light chain variable domain of the parent antibody, more preferably from about 80% to less than 100%, more preferably from about 85% to less than 100%. more preferably from about 90% to less than 100%, and most preferably from about 95% to less than 100%. The antibody variant is generally one which comprises one or more amino acid alterations in or adjacent to one or more hypervariable regions thereof.

A “variant Fc region” comprises an amino acid sequence which differs from that of a native sequence Fc region by virtue of at least one amino acid modification. In certain embodiments, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide, e.g. from about one to about ten amino acid substitutions, and preferably from about one to about live amino acid substitutions in a native sequence Fc region or in the Fc region of the parent polypeptide. The variant Fc region herein will typically possess, e.g., at least about 80% sequence identity with a native sequence Fc region and/or with an Fc region of a parent polypeptide, or at least about 90% sequence identity therewith, or at least about 95% sequence or more identity therewith.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are hound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, e.g., in U.S. Pat. No. 6,194,551 B1 and WO 1999/51642. See also, e.g., Idusogie et al. J. Immunol. 164: 4178-4184 (2000).

An “affinity matured” antibody is one with one or more alterations in one or more CDRs thereof which result an improvement in the affinity of the antibody for antigen, compared to a parent antibody which does not possess those alteration(s). In one embodiment, an affinity matured antibody has nanomolar or even picomolar affinities for the target antigen. Affinity matured antibodies are produced by procedures known in the art. Marks et al. Bio/Technology 10:779-783 (1992) describes affinity maturation by VI-1 and VL domain shuffling. Random mutagenesis of CDR and/or framework residues is described by: Barbas et al. Proc Nat. Acad. Sci. USA 91:3809-3813 (1994); Schier et al. Gene 169:147-155 (1995); Yelton et al. J. Immunol 155:1994-2004 (1995); Jackson et al., J. Immunol. 154(7):3310-9 (1995); and Hawkins et al, J. Mol. Biol. 226:889-896 (1992).

The term “therapeutic antibody” refers to an antibody that is used in the treatment of disease. A therapeutic antibody may have various mechanisms of action. A therapeutic antibody may bind and neutralize the normal function of a target associated with an antigen. For example, a monoclonal antibody that blocks the activity of the of protein needed for the survival of a cancer cell causes the cell's death. Another therapeutic monoclonal antibody may bind and activate the normal function of a target associated with an antigen. For example, a monoclonal antibody can bind to a protein on a cell and trigger an apoptosis signal. Yet another monoclonal antibody may bind to a target antigen expressed only on diseased tissue; conjugation of a toxic payload (effective agent), such as a chemotherapeutic or radioactive agent, to the monoclonal antibody can create an agent for specific delivery of the toxic payload to the diseased tissue, reducing harm to healthy tissue. A “biologically functional fragment” of a therapeutic antibody will exhibit at least one if not some or all of the biological functions attributed to the intact antibody, the function comprising at least specific binding to the target antigen.

“Purified” means that a molecule is present in a sample at a concentration of at least 80-90% by weight of the sample in which it is contained.

The protein, including antibodies, which is purified is preferably essentially pure and desirably essentially homogeneous (i.e. free from contaminating proteins etc.).

An “essentially pure” protein means a protein composition comprising at least about 90% by weight of the protein, based on total weight of the composition, preferably at least about 95% by weight.

An “essentially homogeneous” protein means a protein composition comprising at least about 99% by weight of protein, based on total weight of the composition.

The term “storage-stable” is used to describe a formulation having a shelf-life acceptable for a product in the distribution chain of commerce, for instance, at least 12 months at a given temperature, and preferably, at least 24 months at a given temperature. Optionally, such a storage-stable formulation contains no more than 5% aggregates, no more than 10% dimers, and/or minimal changes in charge heterogeneity or biological activity. Degradation pathways for proteins can involve chemical instability (i.e. any process which involves modification of the protein by bond formation or cleavage resulting in a new chemical entity) or physical instability (i.e. changes in the higher order structure of the protein). Chemical instability can result from, for example, deamidation, racemization, hydrolysis, oxidation, beta elimination or disulfide exchange. Physical instability can result from, for example, denaturation, aggregation, precipitation or adsorption. The three most common protein degradation pathways are protein aggregation, deamidation and oxidation. Cleland et al. Critical Reviews in Therapeutic Drug Carrier Systems 10(4): 307-377 (1993).

As used herein, “soluble” refers to polypeptides that, when in aqueous solutions, are completely dissolved, resulting in a clear to slightly opalescent solution with no visible particulates, as assessed by visual inspection. A further assay of the turbidity of the solution (or solubility of the protein) may be made by measuring UV absorbances at 340 nm to 360 nm with a 1 cm pathlength cell where turbidity at 20 mg/ml is less than 0.05 absorbance units.

“Preservatives” can act to prevent bacteria, viruses, and fungi from proliferating in the formulation, and anti-oxidants, or other compounds can function in various ways to preserve the stability of the formulation. Examples include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of compounds include aromatic alcohols such as phenol and benzyl alcohol, alkyl parabens such as methyl or propyl paraben, and m-cresol. Optionally, such a compound is phenol or benzyl alcohol. The preservative or other compound will optionally be included in a liquid or aqueous form of the CD20 antibody formulation, but not usually in a lyophilized form of the formulation. In the latter case, the preservative or other compound will typically be present in the water for injection (WFI) or bacteriostatic water for injection (BWFI) used for reconstitution.

A “surfactant” can act to decrease turbidity or denaturation of a protein in a formulation. Examples of surfactants include non-ionic surfactant such as a polysorbate, e.g., polysorbates 20, 60, or 80, a poloxamer, e.g., poloxamer 184 or 188, Pluronic polyols, ethylene/propylene block polymers or any others known to the art.

A “biologically functional fragment” of an antibody comprises only a portion of an intact antibody, wherein the portion retains at least one, and as many as most or all, of the functions normally associated with that portion when present in an intact antibody. In one embodiment, a biologically functional fragment of an antibody comprises an antigen binding site of the intact antibody and thus retains the ability to bind antigen. In another embodiment, a biologically functional fragment of an antibody, for example one that comprises the Fc region, retains at least one of the biological functions normally associated with the Fc region when present in an intact antibody, such as FcRn binding, antibody half life modulation, ADCC function and complement binding. In one embodiment, a biologically functional fragment of an antibody is a monovalent antibody that has an in vivo half life substantially similar to an intact antibody. For example, such a biologically functional fragment of an antibody may comprise an antigen binding arm linked to an Fc sequence capable of conferring in vivo stability to the fragment.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

The terms “Protein A” and “ProA” are used interchangeably herein and encompasses Protein A recovered from a native source thereof, Protein A produced synthetically (e.g. by peptide synthesis or by recombinant techniques), and variants thereof which retain the ability to bind proteins which have a C_{H}2/C_{H}3 region, such as an Fc region. Protein A can be purchased commercially from Repligen, Pharmacia and Fermatech. Protein A is generally immobilized on a solid phase support material. The term “ProA” also refers to an affinity chromatography resin or column containing chromatographic solid support matrix to which is covalently attached Protein A.

The term “chromatography” refers to the process by which a solute of interest in a mixture is separated from other solutes in a mixture as a result of differences in rates at which the individual solutes of the mixture migrate through a stationary medium under the influence of a moving phase, or in bind and elute processes.

The term “affinity chromatography” and “protein affinity chromatography” are used interchangeably herein and refer to a protein separation technique in which a protein of interest or antibody of interest is reversibly and specifically bound to a biospecific ligand. Preferably, the biospecific ligand is covalently attached to a chromatographic solid phase material and is accessible to the protein of interest in solution as the solution contacts the chromatographic solid phase material. The protein of interest (e.g., antibody, enzyme, or receptor protein) retains its specific binding affinity for the biospecific ligand (antigen, substrate, cofactor, or hormone, for example) during the chromatographic steps, while other solutes and/or proteins in the mixture do not bind appreciably or specifically to the ligand. Binding of the protein of interest to the immobilized ligand allows contaminating proteins or protein impurities to be passed through the chromatographic medium while the protein of interest remains specifically bound to the immobilized ligand on the solid phase material. The specifically bound protein of interest is then removed in active form from the immobilized ligand with low pH, high pH, high salt, competing ligand, and the like, and passed through the chromatographic column with the elution buffer, free of the contaminating proteins or protein impurities that were earlier allowed to pass through the column. Any component can be used as a ligand for purifying its respective specific binding protein, e.g. antibody.

The terms “non-affinity chromatography” and “non-affinity purification” refer to a purification process in which affinity chromatography is not utilized. Non-affinity chromatography includes chromatographic techniques that rely on non-specific interactions between a molecule of interest (such as a protein, e.g. antibody) and a solid phase matrix.

A “cation exchange resin” refers to a solid phase which is negatively charged, and which thus has free cations for exchange with cations in an aqueous solution passed over or through the solid phase. A negatively charged ligand attached to the solid phase to form the cation exchange resin may, e.g., be a carboxylate or sulfonate. Commercially available cation exchange resins include carboxy-methyl-cellulose, sulphopropyl (SP) immobilized on agarose (e.g. SP-SEPHAROSE FAST FLOW™ or SP-SEPHAROSE HIGH PERFORMANCE™, from GE Healthcare) and sulphonyl immobilized on agarose (e.g. S-SEPHAROSE FAST FLOW™ from GE Healthcare). A “mixed mode ion exchange resin” refers to a solid phase which is covalently modified with cationic, anionic, and hydrophobic moieties, A commercially available mixed mode ion exchange resin is BAKERBOND ABX™ (J. T. Baker, Phillipsburg, N.J.) containing weak cation exchange groups, a low concentration of anion exchange groups, and hydrophobic ligands attached to a silica gel solid phase support matrix.

The term “anion exchange resin” is used herein to refer to a solid phase which is positively charged, e.g. having one or more positively charged ligands, such as quaternary amino groups, attached thereto. Commercially available anion exchange resins include DEAE cellulose, QAE SEPHADEX™ and Q SEPHAROSE™ FAST FLOW (GE Healthcare).

A “buffer” is a solution that resists changes in pH by the action of its acid-base conjugate components. Various buffers which can be employed depending, for example, on the desired pH of the buffer are described in Buffers. A Guide for the Preparation and Use of Buffers in Biological Systems, Gueffroy, D., ed. Calbiochem Corporation (1975). In one embodiment, the buffer has a pH in the range from about 2 to about 9, alternatively from about 3 to about 8, alternatively from about 4 to about 7 alternatively from about 5 to about 7. Non-limiting examples of buffers that will control the pH in this range include MES, MOPS, MOPSO, Tris, HEPES, phosphate, acetate, citrate, succinate, and ammonium buffers, as well as combinations of these.

The “loading buffer” is that which is used to load the composition comprising the polypeptide molecule of interest and one or more impurities onto the ion exchange resin. The loading buffer has a conductivity and/or pH such that the polypeptide molecule of interest (and generally one or more impurities) is/are bound to the ion exchange resin or such that the protein of interest flows through the column while the impurities bind to the resin.

The “intermediate buffer” is used to elute one or more impurities from the ion exchange resin, prior to eluting the polypeptide molecule of interest. The conductivity and/or pH of the intermediate buffer is/are such that one or more impurity is eluted from the ion exchange resin, but not significant amounts of the polypeptide of interest.

The term “wash buffer” when used herein refers to a buffer used to wash or re-equilibrate the ion exchange resin, prior to eluting the polypeptide molecule of interest. Conveniently, the wash buffer and loading buffer may be the same, but this is not required.

The “elution buffer” is used to elute the polypeptide of interest from the solid phase. The conductivity and/or pH of the elution buffer is/are such that the polypeptide of interest is eluted from the ion exchange resin.

A “regeneration buffer” may be used to regenerate the ion exchange resin such that it can be re-used. The regeneration buffer has a conductivity and/or pH as required to remove substantially all impurities and the polypeptide of interest from the ion exchange resin.

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by
the sequence alignment program ALIGN-2 in that program's alignment of A
and B, and
where Y is the total number of amino acid residues in B.
   It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

“Treatment” refers to both therapeutic treatment and prophylactic or preventative measures. Those in need of treatment include those already with the disorder as well as those in which the disorder is to be prevented, “Treatment” herein encompasses alleviation of the disease and of the signs and symptoms of the particular disease.

“Mammal” for purposes of treatment refers to any animal classified as a mammal, including humans, non-human higher primates, other vertebrates, domestic and farm animals, and zoo, sports, or pet animals, such as dogs, horses, cats, cows, etc. Preferably, the mammal is human.

### B. Exemplary Methods and Materials for Carrying Out the Invention

The present invention provides CD20 antibody crystals and methods for recovery and purification of CD20 antibodies. In particular, the invention provides methods, involving crystallization, to recover and purify CD20 antibodies from mixtures in which it is accompanied by other contaminants, such as contaminating proteins and/or other impurities. In a specific embodiment, the invention provides methods to recover and purify CD20 antibodies from recombinant host cultures or cell lysates, such as mammalian cell cultures or cell lysates of CD20 antibody producing E. coli recombinant host cells.

The basis for these purification methods is the identification of conditions under which CD20 antibodies, including antibody fragments, readily crystallize in high purity, and in a size and morphology that allows optimal manipulation throughout the purification process. It has further been found that the purification scheme including a crystallization step is well scaleable, and thus can be used for the large scale purification of CD20 antibodies.

The incorporation of a crystallization step in the purification scheme allows the reduction of purification process steps while maintaining comparable yields to traditional purification schemes using multiple chromatographic purification steps, without crystallization. Accordingly, implementing crystallization into the purification process results in significant savings, without compromising efficiency, yields or product quality.

The practice of the present invention will employ, unless otherwise indicated, conventional techniques of molecular biology and the like, which are within the skill of the art. Such techniques are explained fully in the literature. See e.g., Molecular Cloning: A Laboratory Manual, (J. Sambrook et al., Cold Spring Harbor Laboratory, Cold Spring Harbor, N.Y., 1989); Current Protocols in Molecular Biology (F. Ausubel et al., eds., 1987 updated); Essential Molecular Biology (T. Brown ed., IRL Press 1991); Gene Expression Technology (Goeddel ed., Academic Press 1991); Methods for Cloning and Analysis of Eukaryotic Genes (A. Bothwell et al., eds., Bartlett Publ. 1990); Gene Transfer and Expression (M. Kriegler, Stockton Press 1990); Recombinant DNA Methodology II (R. Wu et al., eds., Academic Press 1995); PCR: A Practical Approach (M. McPherson et al., IRL Press at Oxford University Press 1991); Oligonucleotide Synthesis (M. Gait ed., 1984); Cell Culture for Biochemists (R. Adams ed., Elsevier Science Publishers 1990); Gene Transfer Vectors for Mammalian Cells (J. Miller & M. Calos eds., 1987); Mammalian Cell Biotechnology (M. Butler ed., 1991); Animal Cell Culture (J. Pollard et al., eds., Humana Press 1990); Culture of Animal Cells, 2″ Ed. (R. Freshney et al., eds., Alan R. Liss 1987); Flow Cytometry and Sorting (M. Melamed et al., eds., Wiley-Liss 1990); the series Methods in Enzymology (Academic Press, Inc.); Wirth M. and Hauser H, (1993); Immunochemistry in Practice, 3rd edition, A. Johnstone & R. Thorpe, Blackwell Science, Cambridge, Mass., 1996; Techniques in Immunocytochemistry, (G. Bullock & P. Petrusz eds., Academic Press 1982, 1983, 1985, 1989); Handbook of Experimental Immunology, (D. Weir & C. Blackwell, eds.); Current Protocols in Immunology (J. Coligan et al., eds. 1991); Immunoassay (E. P. Diamandis & T. K. Christopoulos, eds., Academic Press, Inc., 1996); Goding, (1986) Monoclonal Antibodies: Principles and Practice (2d ed) Academic Press, New York; Ed Harlow and David Lane, Antibodies A laboratory Manual, Cold Spring Harbor Laboratory, Cold Spring Harbor, N.Y., 1988; Antibody Engineering, 2^{nd} edition (C. Borrebaeck, ed., Oxford University Press, 1995); and the series Annual Review of Immunology; the series Advances in Immunology.

B.1 Production of CD20 Antibodies

(i) CD20 Antibodies

In various embodiments, the invention provides crystalline forms of 2H7 CD20 antibodies, and methods for the purification of such antibodies, incorporating at least one crystallization step. In specific embodiments, the humanized 2H7 antibody is an antibody listed in Table 1.

TABLE 1
Humanized 2H7 anti-CD20 Antibody and Variants Thereof

| | V_{L} | V_{H} | Full L chain | Full H chain |
|---|---|---|---|---|
| 2H7 | SEQ ID | SEQ ID | SEQ ID | SEQ ID |
| variant | NO. | NO. | NO. | NO. |
| A | 1 | 2 | 6 | 7 |
| B | 1 | 2 | 6 | 8 |
| C | 3 | 4 | 9 | 10 |
| D | 3 | 4 | 9 | 11 |
| F | 3 | 4 | 9 | 12 |
| G | 3 | 4 | 9 | 13 |
| H | 3 | 5 | 9 | 14 |
| I | 1 | 2 | 6 | 15 |

Each of the antibody variants A, B and I of Table 1 comprises the light chain variable sequence (V_{L}):

(SEQ ID NO: 1)
DIQMTQSPSSLSASVGDRVTITCRASSSVSYMHWYQQKPGKAPKPLIYAP
SNLASGVPSRFSGSGSGTDFTLTISSLQPEDFATYYCQQWSFNPPTFGQG
TKVE1KR;
and

the heavy chain variable sequence (V_{H}):

(SEQ ID NO: 2)
EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSS.

Each of the antibody variants C, D, F and G of Table 1 comprises the light chain variable sequence (V_{L}):

(SEQ ID NO: 3)
DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKR,
and

the heavy chain variable sequence (V_{H}):

(SEQ ID NO: 4)
EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSS.

The antibody variant H of Table 1 comprises the light chain variable sequence (V_{L}) of SEQ NO:3 (above) and the heavy chain variable sequence (V_{H}):

(SEQ ID NO: 5)
EVQLVESGGGLVQPGGSLRLSCAASGYTFTSYNMHWVRQAPGKGLEWVGA
IYPGNGATSYNQKFKGRETISVDKSKNTLYLQMNSLRAEDTAVYYCARVV
YYSYRYWYFDVWGQGTLVTVSS

Each of the antibody variants A, B and I of Table 1 comprises the full length light chain sequence:

(SEQ ID NO: 6)
DIQMTQSPSSLSASVGDRVTITCRASSSVSYMHWYQQKPGKAPKPLIYAP
SNLASGVPSRFSGSGSGFDFTLTISSLQPEDFATYYCQQWSFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

Variant A of Table 1 comprises the full length heavy chain sequence:

(SEQ ID NO: 7)
EVQLVESGGGLVQPGGSLRLSCAASGYTFTSYNMHWVRQAPGKGLEWVGA
LYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLINDKSRWQQGNVFSCSVMHEALHNHYTQKSLSLSP
GK.

Variant B of Table 1 comprises the full length heavy chain sequence:

(SEQ ID NO: 8)
EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNKALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK.

Variant I of Table 1 comprises the full length heavy chain sequence:

(SEQ ID NO: 15)
EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVPPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVIINAKTKPREE
QYNATYRVVSVLTVLIIQDWLNGKEYKCKVSNAALPAPIAATISKAKGQP
REPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSL
SPGK.

Each of the antibody variants C. D, F, G and H of Table 1 comprises the full length light chain sequence:

(SEQ ID NO: 9)
DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKRTVAAPSVFIFITSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

Variant C of Table 1 comprises the full length heavy chain sequence:

(SEQ ID N0: 10)
EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTIITCPPCPAPELLGGPSVFLFP
PKPKDTLMISRTPEVTCVVVDVSHEDPEVKFNWYVDGVEVHNAKTKPREE
QYNATYRVVSVLTVLHQDWLNGKEYKCKVSNKALPAPIAATISKAKGQPR
EPQVYTLPPSREEMTKNQVSLTCLVKGFYPSDIAVEWESNGQPENNYKTT
PPVLDSDGSFELYSKLTVDKSRWQQGNVFSCSVMHEALHNHYTQKSLSLS
PGK.

Variant D of Table 1 comprises the full length heavy chain sequence:

(SEQ ID NO: 11)
EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVEPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCAVSNKALPAPIEATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMIIEALHNHYTQKSLSLS
PGK.

Variant F of Table 1 comprises the full length heavy chain sequence:

(SEQ ID NO: 12)
EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK.

Variant G of Table 1 comprises the full length heavy chain sequence:

(SEQ ID NO: 13)
EVQLVESGGGLVQPGGSLRLSCAASGYTFTSYNMHWVRQAPGKGLEWVGA
IYPGNGATSYNQKFKGRETISVDKSKNTLYLQMNSLRAEDTAVYYCARVV
YYSASYWYFDVWGQGTINTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLEPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVESCSVMHEALHWHYTQKSLSLSP
GK.

Variant H of Table 1 comprises the lull length heavy chain sequence:

(SEQ ID NO: 14)
EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSYRYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFELYSKLTVDKSRWQQGNVESCSVMHEALHNHYTQKSLSLSP
GK.

In certain embodiments, the humanized 2H7 antibody further comprises amino acid alterations in the IgG Fc and exhibits increased binding affinity for human FcRn over an antibody having wild-type IgG Fc, by at least 60 fold, at least 70 fold, at least 80 fold, more preferably at least 100 fold, preferably at least 125 fold, even more preferably at least 150 fold to about 170 fold.

The N-glycosylation site in IgG is at Asn297 in the C_{H}2 domain. Humanized 2H7 antibody compositions of the present invention include compositions of any of the preceding humanized 2H7 antibodies having a Fc region, wherein about 80-100% (and preferably about 90-99%) of the antibody in the composition comprises a mature core carbohydrate structure which lacks fucose, attached to the Fc region of the glycoprotein. Such compositions were demonstrated herein to exhibit a surprising improvement in binding to Fc(RIIIA(F158), which is not as effective as Fc(RIIIA (V158) in interacting with human IgG. Fc(RIIIA (F158) is more common than Fc(RIIIA (V158) in normal, healthy African Americans and Caucasians. See Lehrnbecher el al., Blood 94:4220 (1999). Historically, antibodies produced in Chinese Hamster Ovary Cells (CHO), one of the most commonly used industrial hosts, contain about 2 to 6% in the population that are nonfucosylated. YB2/0 and Lec13, however, can produce antibodies with 78 to 98% nonfucosylated species. Shinkawa et al., J Bio. Chem. 278 (5), 3466-347 (2003), reported that antibodies produced in YB2/0 and Lec13 cells, which have less FUT8 activity, show significantly increased ADCC activity in vitro. The production of antibodies with reduced fucose content are also described in e.g., Li et al., (GlycoFi) “Optimization of humanized IgGs in glycoengineered Pichia pastoris” in Nature Biology online publication 22 Jan. 2006; Niwa R. et al., Cancer Res. 64(6):2127-2133 (2004); US 2003/0157108 (Presta); U.S. Pat. No. 6,602,684 and US 2003/0175884 (Glycart Biotechnology); US 2004/0093621, US 2004/0110704, US 2004/0132140 (all of Kyowa Hakko Kogyo).

A bispecific humanized 2H7 antibody encompasses an antibody wherein one arm of the antibody has at least the antigen binding region of the H and/or L chain of a humanized 2H7 antibody of the invention, and the other arm has V region binding specificity for a second antigen. In specific embodiments, the second antigen is selected from the group consisting of CD3, CD64, CD32A, CD16, NKG2D or other NK activating ligands.

The invention also includes purification of other CD20 antibodies, including, without limitation, the therapeutic antibody RITUXAN® (rituximab), which is in clinical practice for the treatment of relapsed or refractory, low-grade or follicular, CD20-positive, B-cell non-Hodgkin's lymphoma (NHL); for the first-line treatment of diffuse large B-cell, CD20-positive, non-Hodgkin's lymphoma (DLBCL- a type of NHL) in combination with CHOP (cyclophosphamide, doxorubicin, vincristine and prednisone) or other anthracycline-based chemotherapy regimens; for the first-line treatment of follicular, CD20-positive, B-cell non-Hodgkin's lymphoma in combination with CVP (cyclophosphamide, vincristine and prednisolone) chemotherapy; and for the treatment of low-grade, CD20-positive, B-cell non-Hodgkin's lymphoma in patients with stable disease or who achieve a partial or complete response following first-line treatment with CVP chemotherapy.

(ii) Antibody production

Monoclonal antibodies, including the CD20 antibodies herein, may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al, Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

In a further embodiment, antibodies or antibody fragments can be isolated from antibody phage libraries generated using the techniques described in McCafferty et al., Nature, 348:552-554 (1990).

Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567: Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iii) Humanized and Human Antibodies

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J.sub.H) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al, Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355; 258 (1992). Human antibodies can also be derived from phage-display libraries (Hoogenboom et al, J. Mol. Biol., 227:381 (1991); Marks et al, J. Mol. Biol., 222:581-597 (1991); Vaughan et al. Nature Biotech 14:309 (1996)). Generation of human antibodies from antibody phage display libraries is further described below.

(iv) Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). In another embodiment as described in the example below, the F(ab′)₂ is formed using the leucine zipper GCN4 to promote assembly of the F(ab′)₂ molecule. According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

(v) Recombinant Production of Antibodies

For recombinant production of an antibody, the nucleic acid encoding it is isolated and inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence (e.g. as described in U.S. Pat. No. 5,534,615, specifically incorporated herein by reference).

Suitable host cells for cloning or expressing the DNA in the vectors herein are the prokaryote, yeast, or higher eukaryote cells described above. Suitable prokaryotes for this purpose include eubactcria, such as Gram-negative or Gram-positive organisms, for example. Enterobacteriaceae such as Escherichia, e.g., E. coli, Enterobacter, Erwinia, Klebsiella, Proteus, Salmonella, e.g., Salmonella typhinurium, Serrafia, e.g, Serratia marcescans, and Shigeila, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X 1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed bySV40 (COS-7, ATCC CRL 1651); human embryonic kidney line (293 or 293 cells subloned for growth in suspension culture, Graham et al, J. Gen Virol. 36:59 (1977)); baby hamster kidney cells (BHK, ATCC CRL 10); Chinese hamster ovary cells/-DHFR(CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC MA 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

In one embodiment, the CD20 antibodies herein are produced in dp12.CHO cells, the production of which from CHO-K1 DUX-B11 cells as described in EP307247. CHO-K1 DUX-B11 cells were, in turn, obtained from CHO-K1 (ATCC No. CCL61 CHO-K1) cells, following the methods described in Simonsen, C. C., and Levinson, A. D., (1983) Proc. Natl. Acad. Sci. USA 80:2495-2499 and Urlaub G., and Chasin, L., (1980) Proc. Natl. Acad. Sci. USA 77:4216-4220, n addition, other CHO-K1 (dhfr) cell lines are known and can be used in the methods of the present invention.

The mammalian host cells used to produce peptides, polypeptides and proteins can be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM, Sigma) are suitable for culturing the host cells. In addition, any of the media described in Ham and Wallace (1979), Meth. in Enz. 58:44, Barnes and Sato (1980), Anal. Biochem. 102:255, U.S. Pat. Nos. 4,767,704; 4,657,866; 4,927,762; or 4,560,655; WO 90/03430; WO 87/00195; U.S. Pat. No. Re. 30,985; or U.S. Pat. No. 5,122,469, the disclosures of all of which are incorporated herein by reference, may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

B.2 Crystallization of C20 Antibodies

Crystallization is widely used for purification of small molecules. However, generally, finding crystallization conditions for proteins, especially for full-length antibodies, where the proper assembly of the three-dimensional antibody structure raises special issues, is very difficult and tedious. Parameters affecting crystallization include, for example, solubility, nucleation and growth rate, and crystal size distribution, each being a function of further parameters, such as temperature, pH, buffer, impurities, and the like. Since antibodies are much more difficult to crystallize than small molecules or small proteins or proteins of simpler structure, the recovery and purification of therapeutic antibodies rarely involves a crystallization step.

B.3 Use of Crystallization in the Recovery and Purification of CD20 Antibodies

In the methods of the present invention, crystallization is a key step in a one-column or a two-column scheme for the recovery and purification of CD20 antibodies.

A protocol for the production, recovery and purification of recombinant antibodies in mammalian, such as CHO, cells may include the following steps:

Cells may be cultured in a stirred tank bioreactor system and a fed batch culture, procedure is employed. In a preferred fed batch culture the mammalian host cells and culture medium are supplied to a culturing vessel initially and additional culture nutrients are fed, continuously or in discrete increments, to the culture during culturing, with or without periodic cell and/or product harvest before termination of culture. The fed batch culture can include, for example, a semi-continuous fed batch culture, wherein periodically whole culture (including cells and medium) is removed and replaced by fresh medium. Fed batch culture is distinguished from simple batch culture in which all components for cell culturing (including the cells and all culture nutrients) are supplied to the culturing vessel at the start of the culturing process. Fed batch culture can be further distinguished from perfusion culturing insofar as the supernate is not removed from the culturing vessel during the process (in perfusion culturing, the cells are restrained in the culture by, e.g., filtration, encapsulation, anchoring to microcarriers etc. and the culture medium is continuously or intermittently introduced and removed from the culturing vessel).

Further, the cells of the culture may be propagated according to any scheme or routine that may be suitable for the particular host cell and the particular production plan contemplated. Therefore, a single step or multiple step culture procedure may be employed. In a single step culture the host cells are inoculated into a culture environment and the processes are employed during a single production phase of the cell culture. Alternatively, a multi-stage culture can be used. In the multi-stage culture cells may be cultivated in a number of steps or phases. For instance, cells may be grown in a first step or growth phase culture wherein cells, possibly removed from storage, are inoculated into a medium suitable for promoting growth and high viability. The cells may be maintained in the growth phase for a suitable period of time by the addition of fresh medium to the host cell culture.

In certain embodiments, fed batch or continuous cell culture conditions may be devised to enhance growth of the mammalian cells in the growth phase of the cell culture. In the growth phase cells are grown under conditions and for a period of time that is maximized for growth. Culture conditions, such as temperature, pH, dissolved oxygen (dO₂) and the like, are those used with the particular host and will be apparent to the ordinarily skilled artisan. Generally, the pH is adjusted to a level between about 6.5 and 7.5 using either an acid (e.g., CO₂) or a base (e.g., Na₂CO₃ or NaOH). A suitable temperature range for culturing mammalian cells such as CHO cells is between about 30° C. to 38° C., and a suitable dO₂ is between 5-90% of air saturation.

At a particular stage the cells may be used to inoculate a production phase or step of the cell culture. Alternatively, as described above the production phase or step may be continuous with the inoculation or growth phase or step.

The cell culture environment during the production phase of the cell culture is typically controlled. Thus, if a glycoprotein is produced, factors affecting cell specific productivity of the mammalian host cell may be manipulated such that the desired sialic acid content is achieved in the resulting glycoprotein. In a preferred aspect, the production phase of the cell culture process is preceded by a transition phase of the cell culture in which parameters for the production phase of the cell culture are engaged. Further details of this process are found in U.S. Pat. No. 5,721,121, and Chaderjian et al., Biotechnol. Prog. 21(4550-3 (2005), the entire disclosures of which are expressly incorporated by reference herein.

Following fermentation proteins are purified. Procedures for purification of proteins from cell debris initially depend on the site of expression of the protein. Some proteins can be caused to be secreted directly from the cell into the surrounding growth media; others are made intracellularly. For the latter proteins, the first step of a purification process involves lysis of the cell, which can be done by a variety of methods, including mechanical shear, osmotic shock, or enzymatic treatments. Such disruption releases the entire contents of the cell into the homogenate, and in addition produces subcellular fragments that are difficult to remove due to their small size. These are generally removed by differential centrifugation or by filtration. The same problem arises, although on a smaller scale, with directly secreted proteins due to the natural death of cells and release of intracellular host cell proteins and components in the course of the protein production run.

Once a clarified solution containing the protein of interest has been obtained, its separation from the other proteins produced by the cell is usually attempted using a combination of different chromatography techniques. These techniques separate mixtures of proteins on the basis of their charge, degree of hydrophobicity, or size. Several different chromatography resins are available for each of these techniques, allowing accurate tailoring of the purification scheme to the particular protein involved. The essence of each of these separation methods is that proteins can be caused either to move at different rates down a long column, achieving a physical separation that increases as they pass further down the column, or to adhere selectively to the separation medium, being then differentially eluted by different solvents. In some cases, the desired protein is separated from impurities when the impurities specifically adhere to the column, and the protein of interest does not, that is, the protein of interest is present in the “flow-through.” Thus, purification of recombinant proteins from the cell culture of mammalian host cells may include one or more affinity (e.g. protein A) and/or ion exchange chomarographic steps.

Ion exchange chromatography is a chromatographic technique that is commonly used for the purification of proteins. In ion exchange chromatography, charged patches on the surface of the solute are attracted by opposite charges attached to a chromatography matrix, provided the ionic strength of the surrounding buffer is low. Elution is generally achieved by increasing the ionic strength (i.e., conductivity) of the buffer to compete with the solute for the charged sites of the ion exchange matrix. Changing the pH and thereby altering the charge of the solute is another way to achieve elution of the solute. The change in conductivity or pH may be gradual (gradient elution) or stepwise (step elution). In the past, these changes have been progressive; i.e., the pH or conductivity is increased or decreased in a single direction.

For further details of the industrial purification of therapeutic antibodies sec, for example, Fahrner et al., Biotechnol. Genet. Eng. Rev. 18:301-27 (2001), the entire disclosure of which is expressly incorporated by reference herein.

A typical protocol for purifying recombinant proteins, such as antibodies, from CHO cell cultures includes the following steps: (1) Protein A chromatography, (2) cation exchange chromatography, (3) viral filtration, (4) anion exchange chromatography and (5) ultrafiltration—diafiltration (UFDF).

Protein A chromatography removes CHO cell proteins (CHOP), CHO cell DNA, gentamycin, insulin, and inactive viral contamination.

Cation exchange chromatography retains biomolecules by the interaction of charged groups that are acidic in nature on the surface of the resin with histidine, lysine and arginine. Cation exchange resins are commercially available from the product lines of various manufacturers, such as, for example. Sigma Aldrich. Cation exchangers include resins carrying, for example, carboxymethyl functional groups (weak cation exchanger, such as, CM cellulose/SEPHADEX® or sulfonic acid functional groups (strong cation exchanger, such as, SP SEPHADEX®). In the second chromatographic purification step of the methods of the present invention, strong cation exchange columns, e.g. SP-SEPHADEX®, SPECTRA/GEL® strong cation exchangers, etc. TSKgel strong cation exchangers, etc. are preferred. In the case of an SP-SEPHAROSE® column, the cross-linked agarose matrix with negatively charged functional groups binds to the CD20 antibody while allowing the majority of the impurities to pass through the column. Elution can be performed using salt gradient elution or step elution, step elution being preferred since it provides better conditions for the subsequent crystallization step, without compromising yields. The elution buffer usually contains sodium chloride or sodium sulfate, and salt concentration is selected to meet the demands of the cation exchange column. The SP-SEPHAROSE® column needs a fairly high salt concentration to remove the bound CD20 protein, while for the subsequent crystallization step relatively low salt concentrations are preferred, in order to lower protein solubility. Typically, about 100-150 mM Na₂SO₄ or 100-200 mM NaCl concentrations are used. A typical elution buffer consists of 200 mM NaCl, 50 mM HEPES, 0.05% Triton X-100, 1 mM DTT, pH 7.5. The cation chromatography step used to remove the remaining CHOP, leached Protein A, remaining CHO DNA, gentamycin, insulin and antibody aggregates.

The viral filtration step provides for high level retrovirus clearance.

Anion exchange chromatography employs resins which are positively charged, e.g. have one or more positively charged ligands, such as quaternary amino groups, attached thereto. Commercially available anion exchange resins include DEAE cellulose, QAE SEPHADEX®. and Q SEPHAROSE Fast Flow® (GE Healthcare). The anion exchange step removes the final remnants of CHOP and CHO DNA and viral impurities, and the UFDF step concentrates and formulates the Q pool.

The present invention provides a purification scheme, in which one or more steps of the traditional purification process are replaced by a crystallization step. Thus, for example, the protein A and subsequent cation exchange purification steps can be replaced by a step of concentrating the HCCF followed by crystallization of the CD20 antibody. The crystallization step effectively removes CHOP, CHO DNA, gentamycin and insulin. In the process including a crystallization step, the CHOP and CHO DNA levels are lower than the corresponding levels after two chromatographic purification steps. In addition, since a Protein A chromatography step is not included, there is no need for the removal of leached Protein A, which results in significant savings. Thus, the new method described herein for the purification of CD20 antibodies from recombinant cell cultures yields a reduction in raw materials and process steps, and yields a highly efficient and scaleable purification scheme, suitable for the large scale production of CD20 antibodies.

While the examples illustrate purification from a mammalian (CHO) cell culture, a similar approach can be applied for the purification of CD20 antibodies from bacterial, e.g. E. coli cells. If the CD20 antibody is produced in E. coli, typically the whole cell broth is harvested and homogenized to break open the E. coli cells and release antibody within the cytoplasm. After removing the solid debris, e.g. by centrifugation, the mixture is loaded onto a cation exchange chromatographic column, such as, for example, SP-Sepharose Fast Flow column (Amersham Pharmacia, Sweden).

In a typical protocol, the pH of the whole cell broth obtained by fermentation of the E. coli cells is adjusted to about 7.5, e.g. by addition of sodium HEPES or any other appropriate buffer. The cells are burst open by one or more passes on a commercially available homogenizer, the cell debris is removed, and the cell lysate is clarified. Specific treatment parameters, such as selection and concentration of reagents, depend on the composition of the starting whole cell broth, such as, for example, cell density. In this cast, the crystallization step might follow cation exchange, e.g. SP-SEPHAROSE® purification. The concentration must be high enough to maximize the solubility differences at different temperatures, but not too high to trigger spontaneous crystallization at or around room temperature.

When crystallization is complete, the CD20 antibody crystals are removed, for example by filtration. The crystals may be kept suspended throughout filtration, using a built-in agitator, or can be deposited in a packed bed. It is important to avoid the formation of a compressed crystal cake, which could make it impossible to achieve the desired flow rate. Flow rates may vary, and typically are between about 200 cm/hr and about 100 cm/hr. The flow rate may depend on the equipment used, and the pressure to be applied during filtration. Filtration may be performed batch-wise or continuously.

Following crystallization and separation, the anti-CD20 antibody crystals can be redissolved and stored or converted into a formulation suitable for the intended use.

Alternatively, a further chromatography purification step can be added to further improve purity by removing the anti-solvent (PEG) residues and buffer components, and reduce the levels of residual extracellular proteins, endotoxin, dimers, and aggregates.

In summary, the purification method for the CD20 binding antibodies of the present invention involves the steps of concentrating the HCCF, crystallizing the antibody under the appropriate conditions, removing and washing the resultant antibody crystals, redissolving the antibody crystals, subjecting the antibody solution to a chromatography purification step, e.g., Q-Sepharose chromatography, and exchanging the purified antibody into the desired formulation using, for example, ultrafiltration/diafiltration.

B.4 Use of Purified Antibodies in Methods of Treatment

The CD20 binding antibodies purified by the methods of the present invention are useful to treat or alleviate an autoimmune disease or a B cell malignancy either as front line therapy or after other treatment, or in conjunction with a second therapeutic agent, either concurrently, sequentially or in alternating regimen. In preferred embodiments the antibody is administered intravenously or subcutaneously.

The methods of treating a CD20 positive, B cell malignancy comprises administering to a patient having the malignancy, a therapeutically effective amount of a CD20 antibody purified by the present methods using crystallization. In specific embodiments, the CD20 antibody is a humanized 2H7 antibody described in Table 1. In specific embodiments the B cell malignancy is a B cell lymphoma or leukemia including non-Hodgkin's lymphoma (NHL), lymphocyte predominant Hodgkin's disease (LPHD), small lymphocytic lymphoma (SLL), chronic lymphocytic leukemia (CLL). Where the B-cell lymphoma is non-Hodgkin's lymphoma (NHL), the NHL includes, but is not limited to, follicular lymphoma, relapsed follicular lymphoma, small lymphocytic lymphoma, mantle cell lymphoma, marginal zone lymphoma, lymphoplasmacytic lymphoma, mycosis fungoides/Sezary syndrome, splenic marginal zone lymphoma, and diffuse large B-cell lymphoma. In some embodiments, the B-cell lymphoma is selected from the group consisting of indolent lymphoma, aggessive lymphoma, and highly aggressive lymphoma. In specific embodiments, humanized CD20 binding antibodies or functional fragments thereof, are used to treat indolent NHL including relapsed indolent NHL and rituximab-refractory indolent NHL.

An “autoimmune disease” herein is a disease or disorder arising from and directed against an individual's own tissues or organs or a co-segregate or manifestation thereof or resulting condition therefrom. In many of these autoimmune and inflammatory disorders, a number of clinical and laboratory markers may exist, including, but not limited to, hypergammaglobulinemia, high levels of autoantibodies, antigen-antibody complex deposits in tissues, benefit from corticosteroid or immunosuppressive treatments, and lymphoid cell aggregates in affected tissues. Without being limited to any one theory regarding B-cell mediated autoimmune disease, it is believed that B cells demonstrate a pathogenic effect in human autoimmune diseases through a multitude of mechanistic pathways, including autoantibody production, immune complex formation, dendritic and T-cell activation, cytokine synthesis, direct chemokine release, and providing a nidus for ectopic neo-lymphogenesis. Each of these pathways may participate to different degrees in the pathology of autoimmune diseases.

“Autoimmune disease” can be an organ-specific disease (i.e., the immune response is specifically directed against an organ system such as the endocrine system, the hematopoietic system, the skin, the cardiopulmonary system, the gastrointestinal and liver systems, the renal system, the thyroid, the ears, the neuromuscular system, the central nervous system, etc.) or a systemic disease which can affect multiple organ systems (for example, systemic lupus erythematosus (SLE), rheumatoid arthritis, polymyositis, etc.). Preferred such diseases include autoimmune rheumatologic disorders (such as, for example, rheumatoid arthritis, Sjögren's syndrome, scleroderma, lupus such as SLE and lupus nephritis, polymyositis/dermatomyositis. cryoglobulinemia, anti-phospholipid antibody syndrome, and psoriatic arthritis), autoimmune gastrointestinal and liver disorders (such as, for example, inflammatory bowel diseases (e.g., ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-negative vasculitis and ANCA-associated vasculitis, including Churg-Strauss vasculitis, Wegener's granulomatosis, and microscopic polyangiitis), autoimmune neurological disorders (such as, for example, multiple sclerosis, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner car disease and hearing loss), Behcet's disease, Raynaud's syndrome, organ transplant, and autoimmune endocrine disorders (such as, for example, diabetic-related autoimmune diseases such as insulin-dependent diabetes mellitus (IDDM), Addison's disease, and autoimmune thyroid disease (e.g., Graves' disease and thyroiditis)). More preferred such diseases include, for example, rheumatoid arthritis, ulcerative colitis, ANCA-associated vasculitis, lupus, multiple sclerosis, Sjögren's syndrome, Graves' disease, IDDM, pernicious anemia, thyroiditis, and glomerulonephritis.

Specific examples of other autoimmune diseases as defined herein, which in some cases encompass those listed above, include, but are not limited to, arthritis (acute and chronic, rheumatoid arthritis including juvenile-onset rheumatoid arthritis and stages such as rheumatoid synovitis, gout or gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, osteoarthritis, arthritis chronica progrediente, arthritis deformans, polyarthritis chronica primaria, reactive arthritis, menopausal arthritis, estrogen-depletion arthritis, and ankylosing spondylitis/rheumatoid spondylitis), autoimmune lymphoproliferative disease, inflammatory hyperproliferative skin diseases, psoriasis such as plaque psoriasis, guttate psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, hives, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, gastrointestinal inflammation, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, graft-versus-host disease, angioedema such as hereditary angioedema, cranial nerve damage as in meningitis, herpes gestationis, pemphigoid gestationis, pruritic scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigcnic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN (RPGN), proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, food allergies, drug allergies, insect allergies, rare allergic disorders such as mastocytosis, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, SLE, such as cutaneous SLE or subacute cutaneous SLE, neonatal lupus syndrome (NLE), and lupus erythematosus disseminatus, juvenile onset (Type I) diabetes mellitus, including pediatric IDDM, adult onset diabetes mellitus (Type II diabetes), autoimmune diabetes, idiopathic diabetes insipidus, diabetic retinopathy, diabetic nephropathy, diabetic colitis, diabetic large-artery disorder, immune responses associated with acute and delayed hypersensitivity mediated by cytokines and T-lymphocytes, tuberculosis, sarcoidosis, granulomatosis including lymphomatoid granulomatosis, agranulocytosis, vasculitides (including large-vessel vasculitis such as polymyalgia rheumatica and giant-cell (Takayasu's) arteritis, medium-vessel vasculitis such as Kawasaki's disease and polyarteritis nodosa/periarteritis nodosa, immunovasculitis, CNS vasculitis, cutaneous vasculitis, hypersensitivity vasculitis, necrotizing vasculitis such as fibrinoid necrotizing vasculitis and systemic necrotizing vasculitis, ANCA-negative vasculitis, and ANCA-associated vasculitis such as Churg-Strauss syndrome (CSS), Wegener's granulomatosis, and microscopic polyangiitis), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia(s), cytopenias such as pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, motoneuritis, allergic neuritis, Behçet's disease/syndrome, Castleman's syndrome, Goodpasture's syndrome, Reynaud's syndrome, Sjögren's syndrome, Stevens-Johnson syndrome, pemphigoid or pemphigus such as pemphigoid bullous, cicatricial (mucous membrane) pemphigoid, skin pemphigoid, pemphigus vulgaris, paraneoplastic pemphigus, pemphigus foliaceus, pemphigus mucus-membrane pemphigoid, and pemphigus erythematosus, epidermolysis bullosa acquisita, ocular inflammation, preferably allergic ocular inflammation such as allergic conjunctivis, linear IgA bullous disease, autoimmune-induced conjunctival inflammation, autoimmune polyendocrinopathies, Reiter's disease or syndrome, thermal injury due to an autoimmune condition, preeclampsia, an immune complex disorder such as immune complex nephritis, antibody-mediated nephritis, neuroinflammatory disorders, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP), post-transfusion purpura (PTP), heparin-induced thrombocytopenia, and autoimmune or immune-mediated thrombocytopenia including, for example, idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, Grave's eye disease (opthalmopathy or thyroid-associated opthalmopathy), polyglandular syndromes such as autoimmune polyglandular syndromes, for example, type I (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant-cell hepatitis, chronic active hepatitis or autoimmune chronic active hepatitis, pneumonitis such as lymphoid interstitial pneumonitis (LIP), bronchiolitis obliterans (non-transplant) vs NSIP, Guillain-Barré syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia such as mixed cryoglobulinemia, amylotrophic lateral sclerosis (ALS; Lou Gehrig's disease), coronary artery disease, autoimmune ear disease such as autoimmune inner car disease (AIED), autoimmune hearing loss, polychondritis such as refractory or relapsed or relapsing polychondritis, pulmonary alveolar proteinosis, keratitis such as Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia areata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, trypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, fibrosing mediastinitis, pulmonary fibrosis, idiopathic pulmonary fibrosis, cystic fibrosis, endophthalmitis, erythema elevatum et diutinum, erythroblastosis fetalis, eosinophilic fasciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis (systemic inflammatory response syndrome (SIRS)), endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis obiterans, thyrotoxicosis, tabes dorsalis, chorioiditis, giant-cell polymyalgia, chronic hypersensitivity pneumonitis, conjunctivitis, such as vernal catarrh, keratoconjunctivitis sicca, and epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders (cerebral vascular insufficiency) such as arteriosclerotic encephalopathy and arteriosclerotic retinopathy, aspermatogenesis, autoimmune homolysis, Boeck's disease, cryoglobulinemia, Dupuytren's contracture, endophthalmia phacoanaphylactica, enteritis allergica, erythema nodosum leprosum, idiopathic racial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica (sympathetic ophthalmitis), neonatal ophthalmitis, optic neuritis, orchitis granulomatosa, pancreatitis, polyradiculitis acuta, pyoderma gangrenosum, Quervain's thyreoiditis, acquired spenic atrophy, non-malignant thymoma, lymphofollicular thymitis, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, autoimmune polyendocrinopathies, oophoritis, primary myxedema, autoimmune atrophic gastritis, rheumatic diseases, mixed connective tissue disease, nephrotic syndrome, insulitis, polyendocrine failure, autoimmune polyglandular syndromes, including polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, allergic sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, spondyloarthropathies, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism such as chronic arthrorheumatism, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, and endometriosis.

B.5 Formulation

For use in the treatment of a disease, a 2H7 antibody purified by the crystallization method of the present invention can be prepared into a liquid formulation comprising the antibody at about 20 mg/ml, 20 mM sodium acetate, 4% trehalose dihydrate, 0.02% polysorbate 20, pH 5.5, for intravenous administration. A liquid formulation comprising humanized 2H7 antibody at about 20 mg/ml, in 20 mM sodium acetate, 240 mM (8%) trehalose dihydrate, pH 5.3, 0.02% Polysorbate 20 is also provided. The 2H7 antibody can also be formulated for subcutaneous administration in a formulation comprising about 150 mg/ml antibody in 30 mM sodium acetate, pH 5.3, 7% trehalose dehydrate, 0.02% polysorbate 20 (Tween 20®).

Further details of the invention are provided in the following non-limiting Examples.

All patents, patent applications, publications, product descriptions, and protocols are cited throughout this application, the disclosures of which are incorporated herein by reference in their entireties.

### EXAMPLES

The following examples are offered for illustrative purposes only, and are not intended to limit the scope of the present invention in any way. Commercially available reagents referred to in the examples were used according to manufacturer's instructions unless otherwise indicated. The source of those cells identified in the following examples, and throughout the specification, by ATCC accession numbers is the American Type Culture Collection, Manassas, Va. In the examples below, 2H7 refers to humanized 2H7 antibody variant A, unless indicated otherwise.

### Example 1

### Dialysis Crystallization Studies

### 1. Effect of PBS Concentration on Dialysis Crystallization of 2H7

Materials and Methods for Dialysis Studies

1. 150 mg/ml 2H7 drug substance

2. Pierce Slide-Alyzer® Dialysis Cassette, 30 kDa cutoff

3, PBS 20× and 1×

4. 1 L glass beaker

The glass beaker with stir bar was filled with 1 L PBS. Per vendor instructions, cassette was presoaked for 30 sec with PBS, then filled with 3 ml of 2H7 using an 18½ gauge needle. The cassette was floated in the beaker and the top was covered with aluminum Coil. Upon end of experiment, the cassette was removed, and any supernatant was removed with an 18½ gauge syringe. The cassette was then cut open along the edge of the membrane, and the remaining material was scrapped off the membrane film using a spatula.

20× and 1×PBS were used to make 0.1×, 1×, 10× and 20× solutions. 150 mg/ml bulk antibody (2H7 drug substance) was dialyzed into beakers containing each PBS concentration. All experiments were performed at 37° C.

Composition of 2H7 drug substance (aka Formulated Bulk):

150 mg/ml 2H7

### 30 mM Sodium Acetate, pH 5.3

7% Trehalose dehydrate

### 0.02% Polysorbate 20

Results

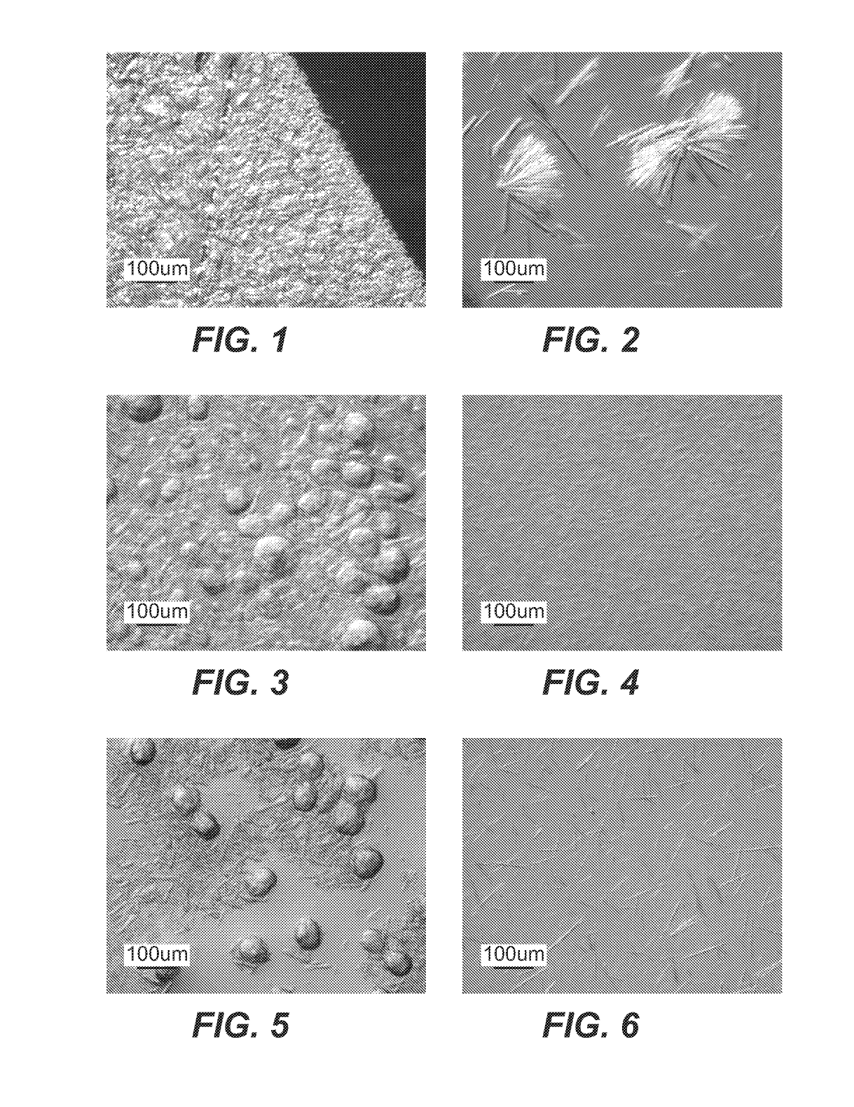

Table 2 shows the visual observations of each cassette after 20 hours. FIG. 1 shows precipitate from the 20× case. Material placed on microscope slides for observation created a thin film that dried and cracked while observing under the microscope (FIG. 2).

TABLE 2
Effect of PBS Concentration on Dialysis Crystallization
of 2H7 bulk @ t = 20 h

| PBS Concentration | Observations |
|---|---|
| 0.1X | No change, no crystallization |
| 1X | 10% of the cassette had small white precipitate. |
| | Cloudiness and long strips of translucent |
| | precipitate as well |
| 10X | Bubbles, totally white and opaque |
| 20X | Thicker than protein in mixture in 10X cassette. |
| | White at the air interface, the rest was milky |
| | and translucent |

### 2. Effect of Temperature on Dialysis Crystallization of 2H7

Based on the previous experiment, 1×PBS was chosen for this study. Experiments were performed at 4° C., 24° C. and 37° C., using a 2-8° C. cold room, room temperature, and incubator environments, respectively. Just as in the previous experiment, crystallization was performed using 150 mg/ml 2H7 bulk.

Results

Table 3 highlights the microscope observations at the end of 24 hours.

TABLE 3
Microscope Observations: Effect of Temperature
on Dialysis Crystallization

| Temperature | % Out of | |
|---|---|---|
| (° C.) | solution | Observations |
| 4 | 100 | A few needle-like crystals (much smaller than |
| | | at 24° C. and 37° C.) in precipitate |
| 24 | 45 | Liquid layer has tiny needles visible to naked |
| | | eye. |
| | | Solid, white layer is amorphous solid with |
| | | small crystals mixed within. Translucent later |
| | | as larger crystals in amorphous precipitate. |
| 36.7 | 45 | Liquid has crystalline needles, |
| | | as seen with 24° C. condition |
| | | in the liquid. Solid, white layer is |
| | | amorphous solid. Translucent layer has |
| | | amorphous solid with cube-like crystals. |

Discussion

It was conjectured that at first, a large amount of protein falls out of solution, which decreases the protein concentration of the solution. At this lower protein concentration, crystals can then form. This appears to be the case in the 24° C. condition, where bands of white precipitate were forming at the same time as translucent layers with what appeared to be crystals. However, under the microscope, it appeared that crystals mixed into the thick opaque white precipitate, but they could not be isolated.

### Example 2

### PBS Batch Studies

Following the dialysis studies described in Example 1, crystallization of 2H7 by direct mixing, also known at the batch method, was studied, using PBS as the precipitant. The experiments were designed to observe the reactions in direct mixing of lower concentrations of 2H7 with PBS at the three temperature points used in the dialysis experiments.

Batch Crystallization

In all batch crystallization studies, a 2H7 CD20 antibody solution was added to a 5-ml tube and allowed to equilibrate at the desired temperature. Precipitant solution (at the same temperature) was added to the tube, and the mixture was rotated continuously in the Lab Quake Tube shaker. At the end of the experiment, (typically 18+ hours), samples were observed under a microscope. The tubes were then centrifuged. The supernatant was sterile filtered and analyzed for antibody concentration.

Materials and Methods

1. 150 mg/ml 2H7 bulk

2. 2H7 buffer without Trehalose/Tween

3. PBS at 20× and 1×

4. 5 ml Falcon tubes BD Falcon Polystrene tubes

5. Pall Acrodisc 13 mm syringe Filters 0.2 μm Supor membrane Pall #4602

6. 5 ml syringe

7. Lab Quake Tube Shaker

8. Microcentrifuge tubes

9. Shimadzu UV/VIS Spectrophotometer

The 2H7 solution was added to the 5 ml tube and allowed to equilibrate at the given temperature. An amount of PBS at the same temperature was added to the 5 ml tube, and the mixture was rotated continuously in the Lab Quake Tube shaker. At the end of the experiment, samples were observed under the microscope. 1 ml samples were transferred from the 5 ml tubes to microcentrifuge tubes and centrifuged for 10 min at 1000 rpm. The supernatant was then filtered using a syringe and 13 mm filter into another microcentrifuge tube. This solution was then diluted accordingly with formulation buffer for UV/VIS analysis.

### 1. Protein to Precipitant Ratio at Three Temperature Points

150 mg/ml 2H7 was diluted to concentrations ranging from 5-100 mg/ml, s using 30 mM sodium acetate buffer. The ratio of 2H7 antibody solution to 1×PBS was varied, and the experiments were performed at 4° C., 24° C., and 37° C. The results were observed over 24 hours.

Results

No changes were observed at any conditions. All tubes remained clear.

Discussion

A higher concentration of PBS might be needed in the case of direct mixing,

### 2. Protein to Precipitant Ratio at Three Temperature Points

The previous experiment was repeated using 10×PBS made from diluting 20×PBS with DI water. Observations were carried till day 4.

Results

Crystals were seen at all temperatures. They varied greatly in size and shape, as noted in Table 4. A summary of Day 4 microscope observations grouped by final 2H7 concentration in the solution is shown in Table 5.

TABLE 4

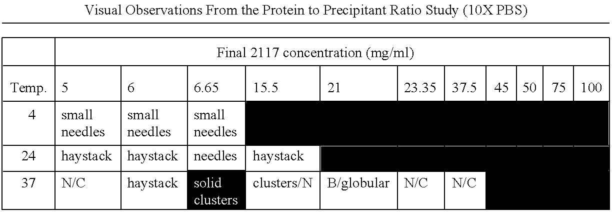

TABLE 5

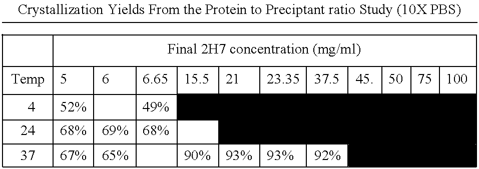

The darkly shaded cells represent the observation of an amorphous solid precipitate. Empty cells represent no change. % values show crystallization efficiency for each condition that resulted in the formation of crystals.

Large “haystack” crystals grown at 24° C. are seen in FIG. 2, FIG. 3 is an example of the irregular heterogeneous crystals seen 37° C. The small needles formed at 4° C. are seen in FIG. 4.

Discussion

Crystallization was most readily seen at 37° C. conditions when the final 2H7 concentration in the tube was <50 mg/ml. At 24° C., large haystack crystals were observed at 6.65 mg/ml and below. These crystals were large, but the crystallization efficiency was low. The results also show that the ratio of PBS to 2H7 solution (v/v) is not very significant. Therefore, for simplicity, it was decided to use a 1:1 ratio of PBS to 2H7 solution (v/v) for future experiments. Temperature was fixed at 37° C. for future experiments, as 2H7 crystallized at this condition at a wide range of concentrations. In addition, this temperature allows for the most flexibility in process design.

### Example 3

### Salt Screening Studies

The purpose of the salt screening studies was to identify other salts that could be used to induce 2H7 crystallization. The starting point was to look at individual salts that compose the PBS buffer. Following these, other salts with similar properties were tested.

Materials and Methods

1. Tris-HCl

2. Tris-Base

3. NaCl

4. Na₂SO₄

5. KCl

6. K₂SO₄

7. Na₂HPO₄

8. KH₂PO₄

9. 30 nM Sodium Acetate buffer

10. 2H7 drug substance

100 ml of 1M stock solutions were prepared in a 20 mM Tris-HCl buffer for each salt. These stock solutions were diluted to the desired concentrations for each experiment using 20 mM Tris-HCL. The final salt concentrations were half of the starting solutions as they were diluted 1:1 when combined with the 2H7 solutions. Five concentration dilutions of 2H7 bulk were made using Sodium Acetate buffer. Crystallization experiments were run at 37° C. using the same procedure as the batch studies described in Example 2. 10×PBS was run as a positive control, and 20 mM Tris-HCl buffer as a negative control,

### 1. Salt Screen

500 mM and 1M made for NaCl, Na2SO4, KCL and K₂SO₄

Results

Some crystallization was seen for KCl and NaCl. Na₂SO₄ cases created a mixture of precipitate and crystals. It appeared that there was crystallization after the majority of the 2H7 fell out of solution as precipitate. This was similar to what was observed in the 10× PBS dialysis experiment. No change was seen with the K₂SO₄. The crystal morphology of 10×PBS crystals varied greatly with the protein concentration. Large round crystals mixed with needles were seen at higher concentrations, while needles were formed at lower concentrations (FIGS. 5 and 6).

### 2. Phosphate Salt Screen

300 mM and 1M solutions were made for KH₂PO₄ and Na₂HPO₄.

Results

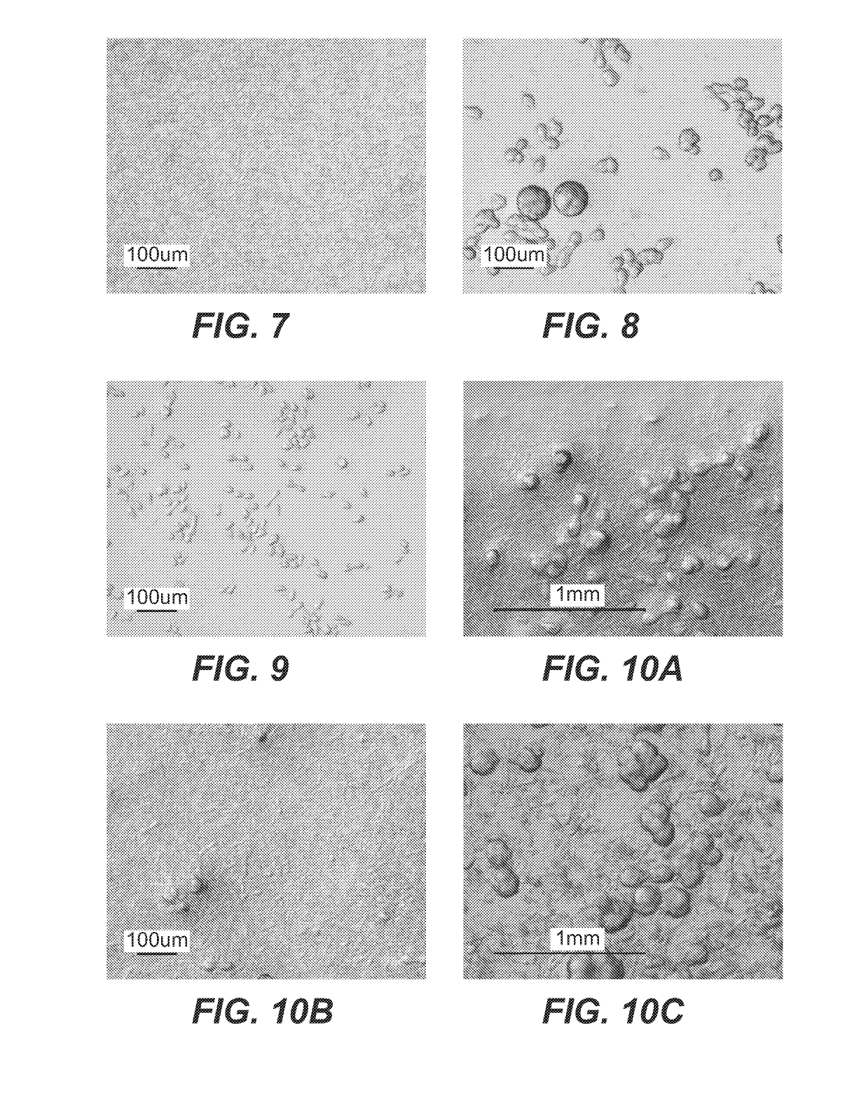

Crystallization was observed using both salts. The Na₂HPO₄ crystals were primarily thinneedles as shown in FIG. 7. The length of the needles was inversely proportional to the 2H7 concentration. A new peanut shape was observed with the KH2PO4 salt (FIG. 8). Some of the peanuts were even clustered in larger globular formations (FIG. 9). This shape is preferable over the thin needles typically seen, because they are thicker, and presumably more robust. The results of microscopic observations are summarized in Table 6. Table 7 shows that the crystallization efficiency of the Na₂HPO₄ cases is much lower than those of KH₂PO₄ experiments.

TABLE 6

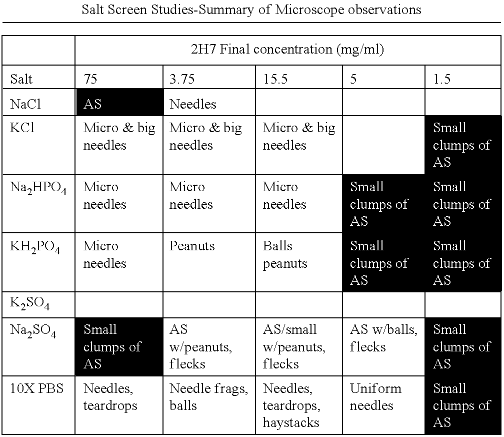

In the foregoing Table, dark shaded cells represent no crystallization, and empty cells represent no change. Grey shade represents a mixture of precipitate and crystals. AS=amorphous solid.

TABLE 7
Salt Screen Studies - Summary of Crystallization Efficiencies

| | | | |
|---|---|---|---|
| NaCl | 1 M | 37.5 | 7 |
| KCl | 1 M | 75 | 84 |
| KCl | 1 M | 15.5 | 52 |
| KCl | 500 mM | 75 | 84 |
| KH₂PO₄ | 300 mM | 75 | 97 |
| KH₂PO₄ | 300 mM | 37.5 | 54 |
| KH₂PO₄ | 300 mM | 17.5 | 77 |
| KH₂PO₄ | 1 M | 75 | 98 |
| KH₂PO₄ | 1 M | 37.5 | 95 |
| KH₂PO₄ | 1 M | 17.5 | 92 |
| Na₂HPO₄ | 300 mM | 75 | 76 |
| Na₂HPO₄ | 300 mM | 37.5 | 54 |
| Na₂HPO₄ | 300 mM | 17.5 | 3 |
| PBS | 10X | 75 | 94 |
| PBS | 10X | 37.5 | 93 |
| PBS | 10X | 17.5 | 88 |
| PBS | 10X | 5 | 59 |

Discussion

Based on crystallization efficiency and range of possible 2H7 concentrations, the results suggest that the best crystallization is seen with KH₂PO₄ and PBS. The 1M KH₂PO₄ conditions yielded >90% crystallization between 75 and 17.5 mg/ml. These crystals had a peanut shape that appears to be more robust than the needles seen with the control. The control, 10×PBS produced largely thin, needle shaped crystals, however crystallization was observed over the largest range of protein concentrations; 75-5 mg/ml. The other individual components of PBS; KCl, Na₂HPO₄ and NaCl did not show similar crystallizing properties, which is unexpected. Upon further investigation, it was noted that the largest component of PBS is NaCl, which exhibited the lowest level of crystallizing properties. Future experiments were designed to look at both PBS and KH₂PO₄ conditions.

### Example 4

### Comparison of 2H7 with and without Trehalose and Polysorbate

The 2H7 used in the previous experiments was from the final 2H7 drug substance, which contains both polysorbate (TWEEN®) 20 and trehalose. It was thought that these components could have a confounding, effect on crystallization. In order to investigate the effects of these components, a pool of 2H7 antibody was concentrated that had been run through the Q-Sepharose chromatography step. This Q-Pool (Q-herein refers to Q-Sepharose) was concentrated using ultrafiltration (UF). Unlike the final bulk material, TWEEN® 20 and trehalose were left out of the formulation. The Q-Step is typically the final chromatography step and precedes concentration and formulation via ultrafiltration/diafiltration (UF/DF). The concentrated Q-pool material was then to be compared side-by-side with the bulk product.

Materials and Methods

1. 2H7Bulk material (2H7 drug substance)

2. 2H7 Concentrated Q-Pool at 169.7 mg/ml

3. Sodium acetate buffer

Both bulk and concentrated material were diluted with sodium acetate buffer to yield the following starting concentrations when combined 1:1 with precipitant:

| D0 | D1 | D2 | D3 | D4 |
|---|---|---|---|---|
| 150 | 37.5 | 17.5 | 5 | 1.5 |

The method described in the batch studies (Example 1) was used to crystallize the protein.

Composition of Q Sepharose pool:
169.7 mg/ml 2H7

### 20 mM Sodium Acetate, pH 5.3

### 1. 10×PBS Screen+/−TWEEN and Trehalose

10×PBS used as precipitant to compare both types of 2H7 at 5 concentrations

### Results

TABLE 8
Effects of TWEEN and trehalose, 1-X PBS crystallization

| Starting Concentration | | |
|---|---|---|
| (mg/ml) | Trehalose/TWEEN | Crystallization efficiency |
| 75 | − | 98% |
| 75 | + | 94% |
| 37.5 | − | 94% |
| 37.5 | + | 94% |
| 17.5 | − | 91% |
| 17.5 | + | 90% |
| 5 | − | 77% |
| 5 | + | 61% |
| 1.5 | − | 4% |
| 1.5 | + | — |

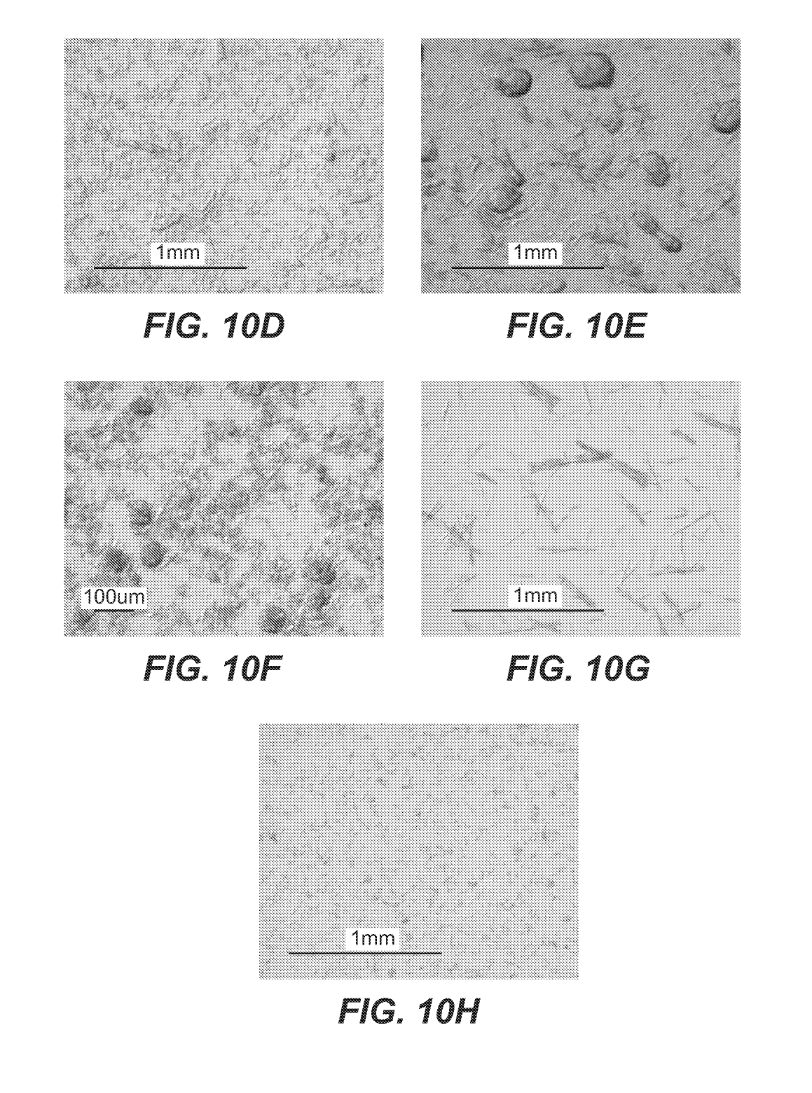

Table 8 highlights the crystallization efficiencies seen in both the presence and absence of TWEEN and trehalose. FIGS. 10A-H compare the crystals morphologies for each concentration.

Discussion

The presence of TWEEN® and trehalose seems to have a negligible effect on crystallization efficiency. This was the first time crystallization was observed at the 1.5 mg/ml concentration. This was only in the case without trehalose and TWEEN®. Since only 4% crystallized, and this was a single experiment, there is not enough evidence to conclusively say that 2H7 without TWEEN®/trehalose crystallizes protein at lower concentrations. The trehalose/TWEEN® does have a significant effect on size and morphology of the crystals. As seen in FIGS. 10A-H, the bulk 2H7 formed bigger crystals at all protein concentrations. Most notably, at 5 mg/ml, long 0.5 mm needles are formed with TWEEN®/trehalose, and 0.05 mm microneedles in the absence of TWEEN®/trehalose,

### 2. KH₂PO₄ Crystallization+/−TWEEN® and Trehalose

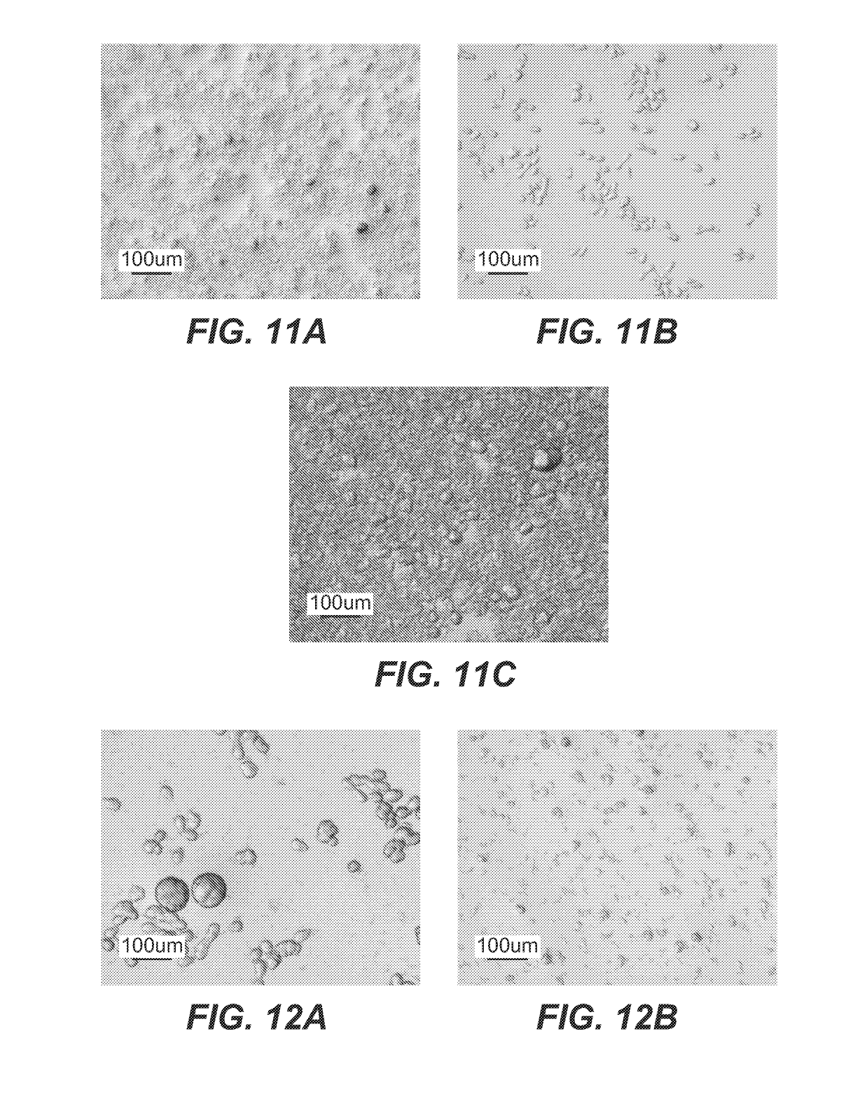

1M KH₂PO₄ solution was used as precipitant to compare both types of 2H7 at 5 concentrations. The results are shown in FIGS. 11A-E. FIGS. 11A-E capture the effects of TWEEN® and trehalose on crystal morphology. Table 9 below shows the differences in crystallization efficiency. The presence of TWEEN® and trehalose had negligible effects on crystallization efficiency, but has a significant effect on size and morphology of crystals. At 37.5 mg/ml, peanut and teardrop shaped crystals were seen in the 2H7 bulk, while the absence of TWEEN® and trehalose resulted in small, mealy, irregular crystals. Precipitation was seen at 500 mM, KH₂PO₄—Trehalose 75 mg/l.

TABLE 9
Effects of TWEEN ® and trehalose, 500 mM KH₂PO₄ crystallization

| Starting Concentration | | |
|---|---|---|
| (mg/ml) | Trehalose/TWEEN | Crystallization efficiency |
| 75 | + | 98% |
| 75 | − | 99% |
| 37.5 | + | 96% |
| 37.5 | − | 98% |
| 17.5 | + | 90% |
| 17.5 | − | 94% |
| 5 | + | 69% |
| 5 | − | 88% |

Summary

The data from this study is conclusive in finding that TWEEN®/trehalose do not affect crystallization efficiency (FIG. 12). The data also suggests that the TWEEN®/trehalose affects the crystal size and morphology. Bigger, more uniform crystals were observed in the presence of TWEEN®/trehalose in both KH2PO4 and 10×PBS cases. Most likely, the TWEEN® is the cause of this difference. Trehalose is a sugar used for cryogenic protection of the protein during freezing and thawing of bulk material. POLYSORBATE® 20 is a nonionic surfactant added to the majority of antibody drug formulations and serves to protect the proteins in these drugs from denaturation and aggregation. Nonionic detergents such as this contain a hydrophobic region which is derived from fatty acid triglycerides. It is possible that the TWEEN® aids in the formation of the crystal lattice which is driven by hydrophobic interactions. It is possible that the morphology and size of crystals can be manipulated by adding TWEEN® or other detergents.

### Example 5

### Crystallization Out of Harvested Cell Culture Fluid (HCCF)

Up until this point, 2H7 hulk and concentrated Q-pool were successfully crystallized. Both of these antibody sources are highly purified. In order to pursue the goal of evaluating feasibility of using crystallization to eliminate one or more chromatography steps in the purification process, crystallization of 2H7 from less purified material was examined. Ideally, 2H7 would be crystallized directly from Harvested Cell Culture Fluid (HCCF). This is the material that enters the purification process after the cell culture process is ended and the cells are removed from the fluid containing secreted 2H7 via centrifugation. If 2H7 could be crystallized from HCCF, we could most likely crystallize the protein at any step in the process. The 2H7 HCCF obtained had a titer of 1.44 mg/ml at the time of harvest. Given that we had not seen consistent crystallization of purified 2H7 at this concentration, some of this material was concentrated using ultrafiltration (UF). The smallest working volume for this equipment was around 500 ml, and we had approximately 10 L of material so we were limited to a maximum concentration of the HCCF. For practical applications, concentration of HCCF much more than 10× would be undesirable due to the time of the operation. Estimating from the Q-pool and bulk experiments, recovery >60% can be expected if the antibody was concentrated to approximately 11×. For these reasons, 10 L of HCCF was concentrated to 15.5 mg/ml.

Materials and Methods

1. 2H7 HCCF concentrated

2. 2H7 HCCF

3. KH₂PO₄

4. PBS

Frozen HCCF was filtered through a 0.2 μm filter after thaw. Dilutions of HCCF were made using the concentrated HCCF and straight HCCF from the same lot. The same batch study method of crystallization was used in this study. At the end of crystallization, protein concentration of the supernatant was measured using Pro Sep A chromatography.

### 1. HCCF Crystallization Proof of Concept Run

1 M and 1.5 M KH₂PO₄ solutions and 20×, 15×, and 10×PBS solutions were used.

HCCF was at 15.5 mg/ml in all cases.

15.5 mg/ml Q-Pool was used as a control.

Results

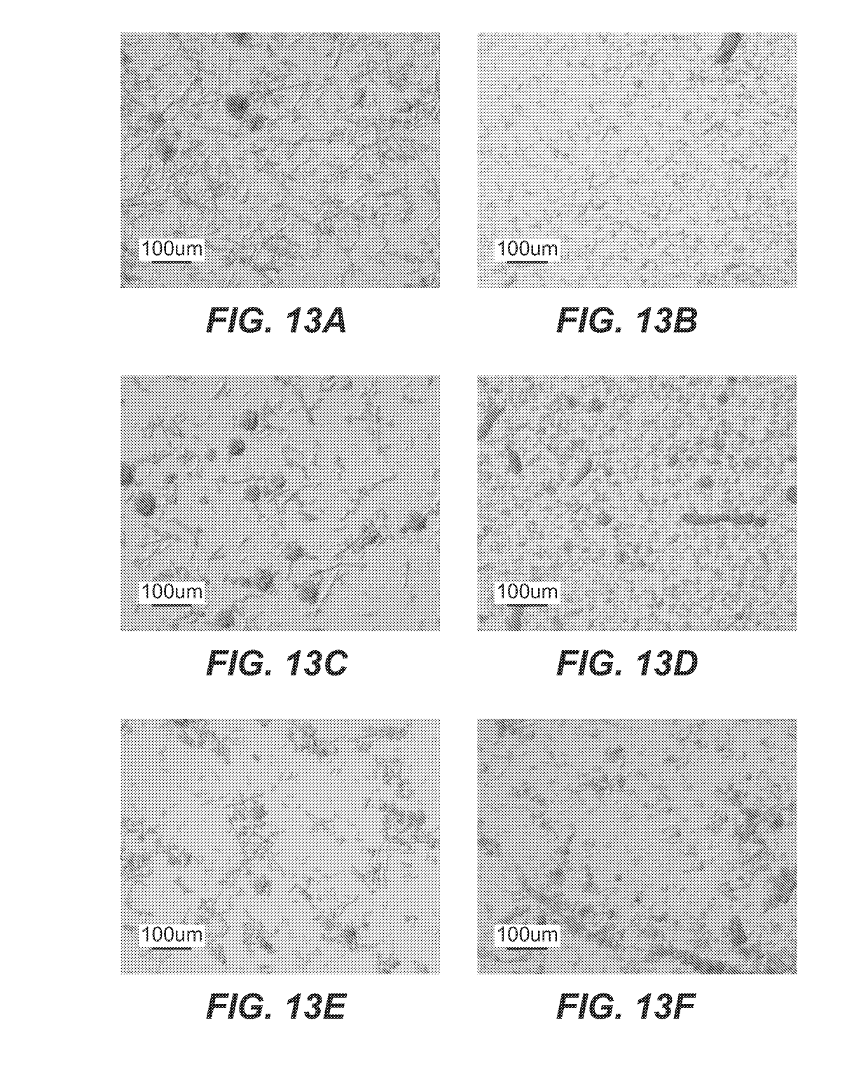

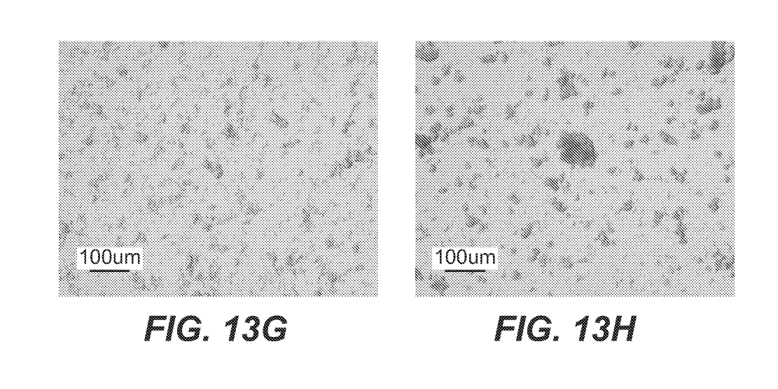

Crystallization was observed at 20×, 15×, and 10×PBS and KH₂PO₄ at both concentrations. The higher PBS concentrations come from stock solutions of PBS buffer that have not been diluted to the typical 1× working concentration. There were noticeable differences in crystal morphology and size (FIGS. 13A-H).

### 2. HCCF Crystallization Screen

HCCF dilutions made between 15.5 and 1.44 mg/ml

2M-200 mM KH₂PO₄ solutions

20×-1×PBS

Results

Crystallization was observed at PBS concentrations ranging from 5× to 20×, and KH2PO4 concentrations between 1.5M and 1M (Table 10).

TABLE 10
HCCF Crystallization Screen

| | Precipitant | 2H7 starting | Crystallization |
|---|---|---|---|
| Precipitant | concentration | concentration | efficiency |
| KH₂PO₄ | 500 mM | 2.5 | 58% |
| KH₂PO₄ | 500 mM | 4.25 | 74% |
| KH₂PO₄ | 500 mM | 7.75 | 98% |
| KH₂PO₄ | 750 mM | 1.5 | 54% |
| KH₂PO₄ | 750 mM | 2.5 | <10% |
| PBS | 5X | 2.5 | 5% |
| PBS | 5X | 4.25 | 70% |
| PBS | 10X | 4.25 | 71% |
| PBS | 10X | 7.75 | 90% |
| PBS | 15X | 2.5 | 70% |
| PBS | 15X | 4.25 | 80% |
| PBS | 15X | 7.75 | 91% |
| PBS | 20X | 2.5 | 77% |
| PBS | 20X | 4.25 | 90% |
| PBS | 20X | 7.75 | 87% |

### 3. HCCF pH Screen

HCCF dilutions made between 15.5 and 1.44 mg/ml

10×PBS solutions prepared at pH, 6, 6.5, 7, 7.7 and 8.

500 mM KH₂PO₄ prepared at pH 6, 6.5, 7, 7.7 and 8.

Results

2H7 will crystallize to varying degrees over pH's ranging from 6.0 and 8.0. The largest range concentrations crystallized with 10×PBS was at pH 7 (Table 11). At pH 7, 0.77 mg/ml, 4.25, and 7.75 mg/ml crystallized, but 1.5 mg/ml did not. This was the first time crystallization was observed at 0.77 mg/ml, which was the non-concentrated HCCF fluid. Crystallization efficiency at that concentration was low, 29%, and this outcome was not repeatable. The low yields and range of crystallization for 10×PBS at pH 6.5 is unusual given that the unadjusted pH is 6.7. Crystallization was achieved over a wide range of pH values and protein concentrations with 500 mM KH₂PO₄. At 7.5 and 8, 2H7 crystallized at the largest range of concentrations, between 1.5 and 7.75 mg/ml (see Table 12). Table 13 highlights the crystal morphologies observed at different concentrations of PBS and KH₂PO₄.

TABLE 11
HCCF pH Screen - 10X PBS

| pH | Initial 2H7 Conc. (mg/ml) | Crystallization Efficiency |
|---|---|---|
| 6 | 4.25 | 57% |
| 6 | 7.75 | 77% |
| 6.5 | 5.25 | 67% |
| 6.5 | 7.75 | 84% |
| 7 | 0.77 | 29% |
| 7 | 4.25 | 70% |
| 7 | 7.75 | 84% |
| 7.5 | 4.25 | 74% |
| 7.5 | 4.25 | 74% |
| 8 | 4.25 | 73% |
| 8 | 7.75 | 82% |

TABLE 12
HCCF pH Screen - 500 mM KH₂PO₄

| pH | Initial 2H7 | Crystallization Efficiency |
|---|---|---|
| 6 | 4.25 | 50% |
| 6 | 7.75 | 88% |
| 6.5 | 4.25 | 81% |
| 6.5 | 7.75 | 84% |
| 7 | 2.5 | 88% |
| 7 | 4.25 | 92% |
| 7 | 7.75 | 92% |
| 7.5 | 1.5 | 83% |
| 7.5 | 2.5 | 88% |
| 7.5 | 4.25 | 93% |
| 7.5 | 7.75 | 94% |
| 8 | 1.5 | 20% |
| 8 | 2.5 | 89% |
| 8 | 4.25 | 77% |
| 8 | 7.75 | 73% |

TABLE 13
Effects of pH on HCCF Crystal Morphology

| pH | 500 mM KH2PO4 | 10X PBS |
|---|---|---|
| 6.0 | Only thin needles at 15.5 mg/ml | Long needles + 100 μm at |
| | | 8.5 mg/ml |
| | | short needles at 15.5 mg/ml |
| 6.5 | Long needles at 8.5 and 15.5 mg/ml | Long needles at 8.5 mg/ml, |
| | | short needles at 15.5 mg/ml |
| 6.7 (Std PBS) | | Needles and needle |
| | | fragments (20-100 μm) at 3-15.5 mg/ml |
| 7.0 | Needles at 5, 8.5, and 15.5 mg/ml | Long needles at 8.5 mg/ml, |
| | | Micro-needles at 15.5 mg/ml |
| 7.2 (Std, KH₂PO₄) | Thick needles and haystacks | |
| | (20-50 μm) at 5, 8.5 and | |
| | 15.5 mg/ml | |
| 7.5 | Short needles at 3 mg/ml, | Needles at 8.5 mg/ml, |
| | irregular clusters at 5, 8.5 | micro-needles at 15.5 mg/ml |
| | and 15.5 mg/ml | |
| 8.0 | Short needles at 1.44 mg/ml, | Needles at 8.5 mg/ml, |
| | irregular clusters, balls and | micro-needles at 15.5 mg/ml |
| | peanuts at 3-15.5 mg/ml | |

Discussion

Comparing overall crystallization efficiencies of the two salts, KH₂PO₄ has consistently higher yields at each pH value. The highest values were seen at 7.5, where all concentrations had yields >80% and 2 cases >92%. In comparison, none of the 10×PBS cases had a yield greater than 84%. Crystallization efficiency increases with protein concentration. With an increase in pH, 500 mM KH₂PO₄ was effective in crystallizing 2H7 at lower concentrations. At pH 6, crystallization was only seen at 4.25 mg/ml and above. At pH 7.5, HCCF crystallized at all concentrations tested with the exception of 1×. It is also interesting to note that there seems to be a peak of effectiveness somewhere between pH 7.5 and 8.0, as noted with a decrease in yields.

The pH also had an effect on the morphology of the crystals. With PBS, needles were seen at all 4.25 and 7.75 mg/ml, however, the needle length was consistently greater at the lower concentration. This suggests that at a lower concentration there is less nucleation and instead more growth and lengthening of existing crystals. Many small crystals are characteristic of a rapid, uncontrolled crystallization process. Looking at KH₂PO₄ conditions, as the pH increased, the morphology of the crystals went from needles to irregular clusters and balls.

### SUMMARY

In this HCCF crystallization study, it was found that concentrated 2H7 readily crystallizes out of HCCF using the same precipitants as identified to crystallize 2H7 bulk and concentrated Q-Pool material. It was not possible to consistently crystallize 2H7 out of un-concentrated HCCF. As with bulk and concentrated Q-pool material, lower crystallization efficiency was seen as the 2H7 concentration decreased. The pH of the precipitant has a significant effect on the crystallization efficiency and concentrations of 2H7 that will crystallize. 500 mM KH₂PO₄ at pH 7.5 showed the highest yields of crystals over the widest range of 2H7 concentrations.

It was also determined that the morphology and size of the 2H7 crystals from HCCF were better than those obtained from concentrated Q-Pool and most comparable to crystals seen with 2H7 bulk. It is possible that the PLURONIC 68 detergent in the HCCF media has an effect similar to the POLYSORBATE 20 found in the bulk material. The morphology of the crystals also varied across the range of precipitant pH values that were explored. Thus, pH is a parameter that can be manipulated to achieve a morphology best suited for downstream processing.

From this study forward, we will use KH₂PO₄ exclusively. From a scale-up perspective, the material requirements for PBS are much greater than that of KH₂PO₄. PBS also contains NaCl at high concentrations which can react with the stainless steel tanks used in manufacturing. We also observed comparable results in the HCCF crystallization proof of concept screen and HCCF crystallization screen. In the HCCF pH screen, we had the highest efficiencies, and greatest range of pH flexibility and morphology with KH₂PO₄. In the following studies, we look at further optimizing the precipitant conditions by looking at tighter pH ranges and developing phase maps of the crystallization process.

### Example 6

### Further Analysis of Process Parameters for Harvested Cell Culture Fluid (HCCF) Process

After determining that 2H7 would readily crystallize from HCCF, the focus has been shifted from bulk and Q-pool material to crystallization from concentrated HCCF. From a purification standpoint, it would be most useful to replace some of the expensive and time and labor intensive upstream processes, like the Protein A purification step (Pro-A), with crystallization. The goal of this study is to further refine the precipitant conditions.

Materials and Methods

### KH₂PO₄

### Concentrated 2H7 HCCF

2H7 HCCF from pH Optimization
500 mM KH₂PO₄ solutions at 6 points between 7 and 8
1.44 mg-8.5 mg/ml 2H7 concentrations
Run in duplicate

### Results

Crystallization was observed over a wide range of concentrations. The highest crystallization efficiencies were seen at the 8.5 mg/ml HCCF 2H7 concentration.

TABLE 14
HCCF pH Optimization

| Starting 3H7 | | | |
|---|---|---|---|
| Concentration | | Crystallization | |
| (mg/ml) | KH₂PO₄ pH | efficiency | Standard deviation |
| 1.5 | 7.4 | 66% | 0.23 |
| 1.5 | 7.6 | 73% | 0.06 |
| 1.5 | 7.8 | 78% | 0.05 |
| 1.5 | 8 | 81% | 0.01 |
| 2.5 | 7 | 71% | 0.22 |
| 2.5 | 7.2 | 69% | 0.05 |
| 2.5 | 7.4 | 81% | 0.08 |
| 2.5 | 7.6 | 84% | 0.03 |
| 2.5 | 7.8 | 87% | 0.04 |
| 2.5 | 8 | 86% | 0.01 |
| 4.25 | 8 | 92% | 0.00 |
| 4.25 | 7.2 | 81% | 0.02 |
| 4.25 | 7.4 | 85% | 0.03 |
| 4.25 | 7.6 | 90% | 0.03 |
| 4.25 | 7.8 | 90% | 0.02 |
| 4.25 | 8 | 91% | 0.00 |
| 7.75 | 7 | 94% | |
| 7.75 | 7.2 | 88% | 0.01 |
| 7.75 | 7.4 | 91% | 0.01 |
| 7.75 | 7.6 | 92% | 0.01 |
| 7.75 | 7.8 | 94% | 0.01 |
| 7.75 | 8 | 95% | 0.00 |

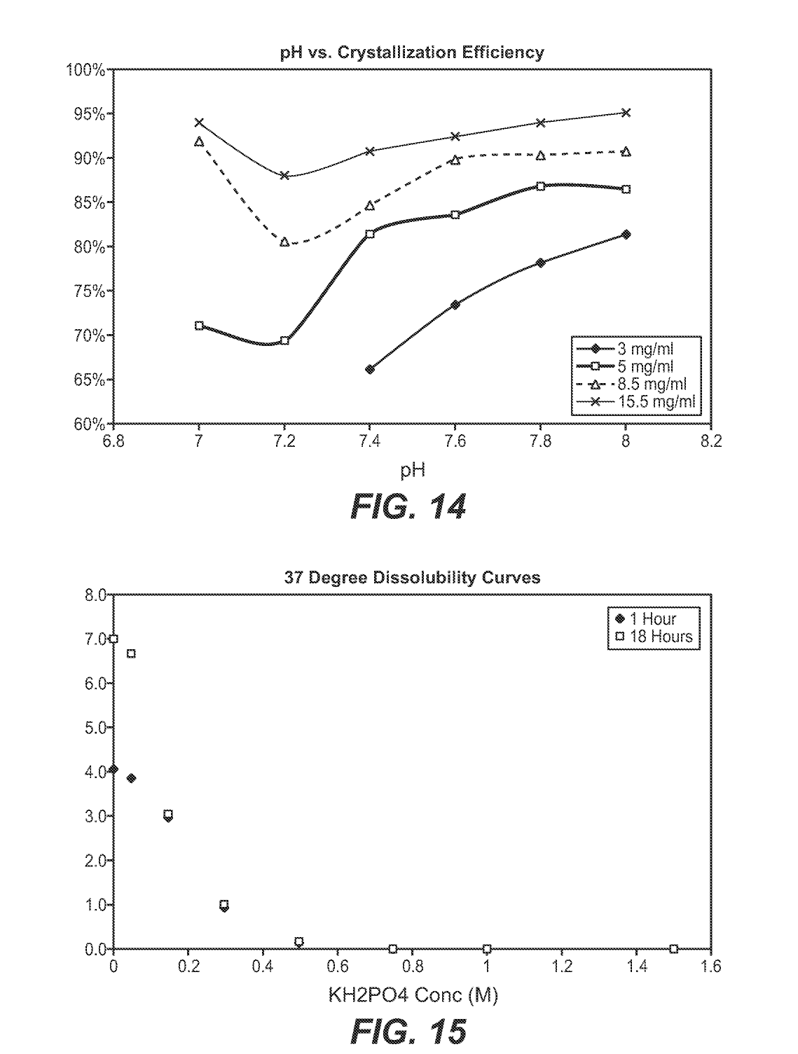

A graphic illustration of the HCCF crystallization efficiency as a function of pH, using 500 mM KH₂PO₄, is shown in FIG. 14.

Discussion

As seen in FIG. 14, at 2.5 mg/ml 2H7 and above, there is little difference in crystallization efficiency between pH 7.6 and 8.0. The maximum and decline of crystallization efficiency between pH 7.5 and 8 was not observed in this experiment. 2H7 at a concentration of 1.5 mg/ml, did not crystallize at 7.0, and 7.2. There was also a significant increase in crystallization efficiency as the pH increased to 8.0. The final pH in each tube once the KH₂PO₄ and HCCF were combined was consistently between 0.2-0.3 less than that of the KH₂PO₄ solution added.

Summary

Based on these experiments, for crystallization from CCF, the optimal pH of K2PO4 is 7.8+/−0.2, and the optimal concentration of the salt is 1 M. However, as the data show, other pHs and concentrations also work.

### Example 7

### Dissolubility Studies

From the studies described in the previous Examples, the regions have been determined in which precipitation and nucleation of crystals are observed. Here, we will determine the metastable region, where one no longer gets new crystal formation, but instead sees crystal growth. To this end, we will determine the conditions where crystals begin to dissolve back into solution. The dissolubility studies aim at determining factors of KH₂PO₄ pH and concentrations and developing solubility curves that will complete the crystallization phase maps for 2H7.

Materials and Methods

1. 2H7 concentrated HCCF

### 2. 2H7 HCCF

### 3, KH₂PO₄

Crystals were prepared using the large batch method. The supernatant from the tubes was removed using a benchtop aspirator. Approximately 50 ml of KH₂PO₄ at pH 7.2 was added to the falcon tube which was then shaken to resuspend the crystals in the salt solution. This mixture was then centrifuged again and this supernatant was exchanged for fresh KH₂PO₄. This process was repeated 2×.

After the third resuspension in KH₂PO₄, 2 ml of this mixture was placed into 5 ml falcon tubes. The tubes were centrifuged once, and the supernatant was aspirated and replaced with 2 ml of KH₂PO₄ at the desired condition.

These tubes were placed in the rotator, and left 18+ hours. At the end of this time, approximately 1 ml of the mixture was filtered through a syringe with 13 mm filter into a microcentrifuge tube. The protein concentration in solution was measured using Pro A analysis.

### 1. KH₂PO₄ Concentration Dissolubility Study

KH₂PO₄ solutions at 7.8 ranging from 0.150M to 1.5M
Water used as a control condition
Concentrations measured at 1 h and 18 h
4° C., Ambient temperature 24° C., and 37° C.

Results

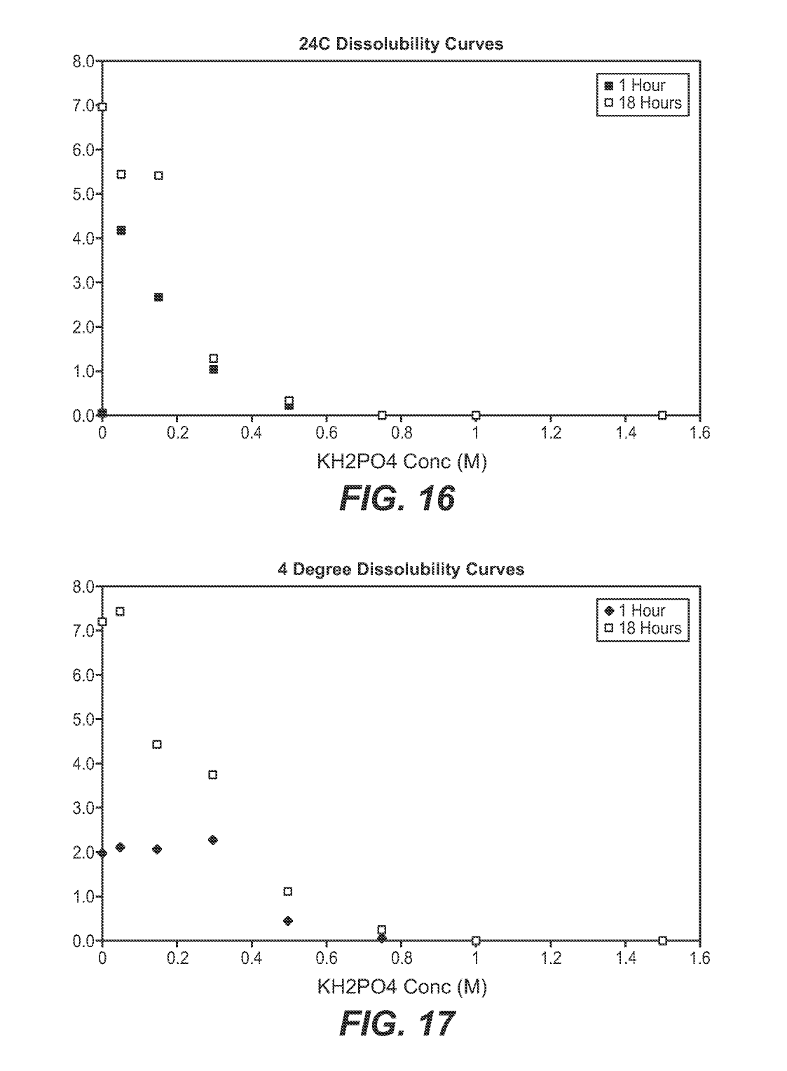

The dissolubility curves are shown in FIG. 15-17.

Discussion

It was found that crystals redissolved in solutions at 750 mM and below. It appears that this takes place at the fastest rate when incubated at 4° C. with 26.7% dissolving within the 1^{st} hour. This is consistent with our understanding that crystallization is optimal at higher temperature. There seems to be little difference between rates and percentages of dissolution for 24° C. and 37° C.

### Example 8

### Purification Starting from Concentrated HCCF with Crystallization Step

Based on the previous experiments, the basic steps of the crystallization unit operation, i.e. concentration, crystallization, washing and dissolution, can replace two chromatography steps. In this example, the starting material is concentrated HCCF, which is run through this new 2H7 purification process. Product purity and quality data are then collected and compared with the traditional purification process.

Materials and Methods

### 1. Concentrated 2H7 HCCF

### 2. 1M K2HPO4

3. 250 ml flask
4. Q-Sepharose buffer
5. Q-Sepharose column

### 6. Centriprep

70 ml of 2H7 HCCF at 15 g mg/ml crystallized in 250 ml flask with 750 ml K2PO4 using large batch method. Crystals were washed and dissolved into Q-Pool buffer using the optimized processes. Samples were taken. The 2H7 pool purified using a Q-Sepharose column at the given process conditions and samples were taken. The Q-pool was concentrated using Centri-prep and samples were taken. Samples were analyzed for titer, CHOP levels and aggregates.

Results

The centriprep was used to concentrate the antibody because we were concentrating a small volume of material, <1 L. For a volume greater than 1 L, a benchtop TFF can be used. Since the UF/DF generally has little effect on the purity and quality of the final product, the centriprep was seen as an acceptable substitute.

TABLE 15
Purification Process Comparison - Conventional vs. Crystallization Purity Levels

| | | | CHO | SEC | | |
|---|---|---|---|---|---|---|
| 2H7/12K | CHOP | LpA | DNA | (% Agg) | Gentamycin | Insulin |
| feedstock | ng/mg | ng/mg | ng/mg | (HMWS) | ng/mg | ng/mg |
| HCCF | 340,000-540,000 | N/A | 2500-4000 | N/A | 16,000-26,000 | 0.1 to 30 |
| Prosep vA | 1900-3100 | 8-13 | 1-2 | 1.1-1.3 | 7-13 | LTD |
| SP | 550-650 | <2 | 0.001-0.02 | 0.8-1.1 | LTD | LTD |
| SEPHAROSE | | | | | | |
| FF | | | | | | |
| Q | 6-10 | <2 | LTD | 0.6-0.7 | LTD | LTD |
| SEPHAROSE | | | | | | |
| FF | | | | | | |
| UFDF | 3-6 | <2 | LTD | 0.7-0.8 | LTD to 0.03 | LTD |
| HCCF | 340,000-540,000 | N/A | 2500-4000 | N/A | 16,000-26,000 | 0.1 to 30 |
| Concentrated | 3.4-5.4 | 8013 | 10-20 | ? | 160,000-260,000 | 1 to 300 |
| HCCF | million | | | | | |
| Crystallization | 150-250 | N/A | LTD | 2.1-2.2 | LTD | LTD |
| Q | 6-10 | <2 | LTD | 0.6-0.7 | LTD | LTD |
| SEPHAROSE | | | | | | |
| FF | | | | | | |
| Centriprep | 3-6 | <2 | LTD | 0.7-0.8 | LTD | LTD |

% Fragment (LMWS): 0.2-0.3%

Summary

The process was successful in purifying 2H7. The CHOP levels were within the range of the standard process. The aggregate levels were higher than in the current process, however, they were within range of the Certificate Analysis for 2H7. This is, at least in part, due to removing the SP-SEPHAROSE® step, which serves to remove aggregates. It may be possible to optimize the Q-SEPHAROSE® step to remove aggregates. Higher aggregates may also be due to the shear rates for concentrating 2H7 HCCF. UF will be investigated for any effect on aggregates.

### Example 9

### Effect of Potassium Phosphate Concentration on Crystallization of Hu 2H7 Variant H

Tests were conducted with purified 2H7 variant H to see if it would crystallize in similar conditions to 2H7 variant A (see Table 1).

Materials and Methods

1. 2H7 variant H unconditioned Bulk at 23 mg/ml (Material that has been concentrated and diafiltered but has not had trehalose or Tween™ added)

2. 1 M Potassium Phosphate, pH 7.8

3. Purified Water

The water and the potassium phosphate were mixed to make a series of phosphate concentrations from 0-1.0 M. These along with the unconditioned bulk were heated to 37° C. then mixed 1:1, and incubated at 37° C. with mixing for 24 hours. Samples were then centrifuged and the supernatants assayed for remaining 2H7 variant H concentration.

Results

TABLE 16
Effects of Potassium Phosphate Concentration on
Crystallization of 2H7 variant H

| Potassium Phosphate | |
|---|---|
| Concentration (mM) | Crystallization efficiency |
| 0 | 0% |
| 50 | 32.7% |
| 100 | 71.2% |
| 150 | 93.4% |
| 200 | 96.6% |
| 250 | 98.2% |
| 300 | 99.0% |
| 350 | 99.2% |
| 400 | 99.6% |
| 450 | 99.2% |
| 500 | 99.8% |

Discussion

This experiment confirmed that the conditions discovered for 2H7 variant A are applicable to 2H7 variant H. Although 2H7 variant H showed similar crystallization behavior to variant A, it achieved a near 100% crystallization efficiency at only 250 mM potassium phosphate at pH 7.8.

### Example 10

### Effect of Potassium Phosphate Concentration on Crystallization of Variant C

Tests were conducted with purified Variant C to see if it would crystallize in similar conditions to 2H7 variant A.

Materials and Methods

1. Variant C unconditioned Bulk at 25.3 mg/ml (material that has been concentrated and diafiltered but has not had trehalose or Tween added)

2. 1 M Potassium Phosphate, pH 7.8

3. Purified Water

The water and the potassium phosphate were mixed to make a series of phosphate concentrations from 0-1.0 M. These along with the unconditioned bulk were heated to 37° C. then mixed 1:1, and incubated at 37° C. with mixing for 24 hours. Samples were then centrifuged and the supernatants assayed for remaining variant C concentration.

Results

TABLE 17
Effects of Potassium Phosphate Concentration on
Crystallization of variant C

| Potassium Phosphate | |
|---|---|
| Concentration (mM) | Crystallization efficiency |
| 0 | 0% |
| 50 | 26.9% |
| 100 | 31.9% |
| 150 | 83.8% |
| 200 | 91.6% |
| 250 | 95.8% |
| 300 | 97.3% |
| 350 | 98.3% |
| 375 | 98.8% |
| 400 | 99.1% |
| 450 | 99.6% |
| 500 | 99.7% |

Discussion

This experiment confirmed that the conditions discovered for humanized 2H7 variant A are applicable to variant C. Although variant C showed similar crystallization behavior to variant A, it achieved a near 100% crystallization efficiency at only 300 mM potassium phosphate at pH 7.8.

### Example 11

### Effect of Potassium Phosphate Concentration and pH on Crystallization of Variant C from Concentrated HCCF

Tests were conducted with variant C concentrated HCCF to determine the effect of pH and phosphate concentration on variant C crystallization and the resulting purification.

### Materials and Methods

1. variant C concentrated HCCF at 13.8 mg/ml, 1.8×10⁶ ng/mg host cell protein

2. 1 M Potassium Phosphate, pH3, pH 4, pH5, pH 6, pH7, pH 8

3. Purified Water

The water and the potassium phosphate were added to the concentrated HCCF at 37° C. to make a series of crystallization experiments at phosphate concentrations between 0.2 and 0.5 M. These were done in 2 groups, the variant C concentration was kept consistent in each, and hence the highest phosphate concentration in each group determined final dilution for that group. After mixing and incubation for greater than 24 hours, the samples were centrifuged and the supernatants were measured for residual variant C. For the samples from the first group, the crystals were dissolved and the variant C and host cell protein concentrations were measured to assess the purity after crystallization.

TABLE 18
Effect of Potassium Phosphate Concentration and pH on
Crystallization of variant C from Concentrated HCCF

| | | variant C starting | | |
|---|---|---|---|---|
| | Potassium | concentration | Crystallization | HCP |
| pH | Phosphate | (mg/ml) | efficiency | ng/mg |
| 8 | 300 mM | 8.35 | 84.0% | 133.0 |
| 8 | 363 mM | 8.35 | 92.0% | 292.0 |
| 8 | 417 mM | 8.35 | 94.5% | 322.0 |
| 7 | 300 mM | 8.35 | 73.5% | 196.0 |
| 7 | 363 mM | 8.35 | 84.6% | 275.0 |
| 7 | 417 mM | 8.35 | 91.3% | 210.0 |
| 6 | 300 mM | 8.35 | 0.0% | n/a |
| 6 | 363 mM | 8.35 | 48.2% | 63.2 |
| 6 | 417 mM | 8.35 | 68.3% | 121.0 |
| 5 | 200 | 6.9 | 3.6% | n/a |
| 5 | 300 | 6.9 | 1.7% | n/a |
| 5 | 400 | 6.9 | 8.3% | n/a |
| 5 | 500 | 6.9 | 6.7% | n/a |
| 4 | 200 | 6.9 | 0.1%* | n/a |
| 4 | 300 | 6.9 | 27.6%* | n/a |
| 4 | 400 | 6.9 | 25.2%* | n/a |
| 4 | 500 | 6.9 | 24.3%* | n/a |
| 3 | 200 | 6.9 | 97.3%* | n/a |
| 3 | 300 | 6.9 | 100%* | n/a |
| 3 | 400 | 6.9 | 100%* | n/a |
| 3 | 500 | 6.9 | 100%* | n/a |

*precipitation

### Discussion

Like 2H7 variant A, the concentration of phosphate required to induce crystallization is reduced with increasing pH. At pH 5, very little crystallization was observed at the phosphate concentrations used. At pH 3 and 4, variant C precipitated rather than crystallized. Precipitation differs from crystallization in that it happens instantly upon mixing, the solid produced does not settle, and cannot be redissolved. The level of host cell protein was reduced to less than 0.2% of the starting material in all the cases measured indicating that crystallization is an effective purification tool.

### Example 12

### Application of Crystallization to the Purification of Variant C from HCCF

Since variant C did not crystallize at pH 5 at potassium phosphate concentrations that induced crystallization at higher pH, diafiltration with 0.4 M potassium phosphate pH 5.0 was incorporated into the initial HCCF concentration step. Crystallization was then induced by adjusting concentrated HCCF to pH 7.8.

### Materials and Methods

1. variant C HCCF at 1.8 mg/ml

2. 0.4 M Potassium Phosphate, pH5

3. Millipore Ultrafiltration Unit

A 10 L aliquot of variant C HCCF were concentrated ˜10 fold by ultrafiltration. It was then diafiltered with 5 diavolumes of 0.4 M Potassium Phosphate pH 5.0. The concentrated variant C HCCF was recovered from the system, adjusted to 37° C. and subsequently to pH 7.8. The sample was incubated at 37° C. with gentle mixing for 46 hours at which time the crystals were recovered by centrifugation. Each batch of crystals was washed twice with 0.4 M Potassium phosphate pH 8, then dissolved in 25 mm Tris pH 8. The crystal pool had to be adjusted to pH 5.5 to achieve complete dissolution.

### Results

TABLE 19
Purification Results

| Step | Yield (%) | Host Cell Protein (ng/mg) |
|---|---|---|
| HCCF | 100.0 | 87741.6 |
| Concentrated HCCF | 103.5 | 176856.6 |
| Supernatant | 7.7 | 748543.3 |
| Wash 1 | 2.0 | 90226.9 |
| Wash 2 | 0.7 | 42683.2 |
| Dissolved Crystals | 76.1 | 904.7 |

### Discussion

The above crystallization procedure removed 99% of the host cell proteins from the starting variant C HCCF. The yield of 76% is comparable to the standard antibody process. Crystallizing the antibody by diafiltration into the crystallization solution rather than by direct addition of the crystallization solution to the antibody solution allows maintenance of higher antibody concentrations during crystallization. Diafiltration accomplishes two functions—it is a method of exchanging into a different buffer, in this case, from HCCF into the crystallization buffer comprising the desired salt and pH, while at the same time concentrating the HCCF solution. Because the concentration of soluble antibody at the end of crystallization is independent of the starting concentration, starting with higher antibody concentrations increases the potential yield. Exchanging into the crystallization buffer by diafiltration is especially useful when the required concentration of crystallizing agent is near the solubility limit of the agent. For example, it would be impossible to conduct crystallization at the solubility of potassium phosphate by diluting the antibody solution with concentrated potassium phosphate, but this is achievable via diafiltration.

Conclusions

We have demonstrated that 2H7, a humanized monoclonal antibody, and its variants can be crystallized. It has been determined that the crystallization is optimal at increased temperatures (4-40 C), in the presence of KH₂PO₄. 2H7 has been crystallized from concentrated, purified bulk, concentrated Q-Pool and concentrated HCCF. By optimizing the process conditions, crystallization efficiencies of over 90% could be achieved from concentrated HCCF. Because of the high level of purity, HCCF crystallization can replace two of the longest, most expensive chromatography steps, Protein A chromatography and SP-SEPHAROSE chromatography. This was directly proven by purifying 1 gram of 2H7. The final product was comparable to the product seen with the traditional process. Thus, crystallization is a feasible process step for purifying 2H7 and its variants.

While the experiments were conducted with specific CD20 antibodies, the humanized 2H7 antibody variants, this approach is equally suitable for crystallizing other CD20 antibodies, including, without limitation, Rituximab (RITUXAN®), and the 2H7 variants specifically disclosed herein.

The invention illustratively described herein can suitably be practiced in the absence of any element or elements, limitation or limitations that is not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalent of the invention shown or portion thereof, but it is recognized that various modifications are possible within the scope of the invention claimed. Thus, it should be understood that although the present invention has been specifically disclosed by preferred embodiments and optional features, modifications and variations of the inventions embodied herein disclosed can be readily made by those skilled in the art, and that such modifications and variations are considered to be within the scope of the inventions disclosed herein. The inventions have been described broadly and generically herein. Each of the narrower species and subgeneric groupings falling within the generic disclosure also form the part of these inventions. This includes within the generic description of each of the inventions a proviso or negative limitation that will allow removing any subject matter from the genus, regardless or whether or not the material to be removed was specifically recited. In addition, where features or aspects of an invention are described in terms of the Markush group, those schooled in the art will recognize that the invention is also thereby described in terms of any individual member or subgroup of members of the Markush group. Further, when a reference to an aspect of the invention lists a range of individual members, as for example, “SEQ ID NO:1 to SEQ ID NO:100, inclusive,” it is intended to be equivalent to listing every member of the list individually, and additionally it should be understood that every individual member may be excluded or included in the claim individually.

From the description of the invention herein, it is manifest that various equivalents can be used to implement the concepts of the present invention without departing from its scope. Moreover, while the invention has been described with specific reference to certain embodiments, a person of ordinary skill in the art would recognize that changes can be made in form and detail without departing from the spirit and the scope of the invention. The described embodiments are considered in all respects as illustrative and not restrictive. It should also be understood that the invention is not limited to the particular embodiments described herein, but is capable of many equivalents, rearrangements, modifications, and substitutions without departing from the scope of the invention. Thus, additional embodiments are within the scope of the invention and within the following claims. All U.S. patents and applications; foreign patents and applications; scientific articles; books; and publications mentioned herein are hereby incorporated by reference in their entirety as if each individual patent or publication was specifically and individually indicated to be incorporated by reference, including any drawings, figures and tables, as though set forth in full.

What is claimed is:

1. A method of purifying a CD20 antibody from a mixture, comprising crystallizing the CD20 antibody and recovering the CD20 antibody from said mixture.

2. The method of claim 1 wherein said mixture has not been subjected to prior lyophilization.

3. The method of claim 1 wherein said mixture is concentrated Harvested Cell Culture Fluid (HCCF) of a recombinant cell culture producing the CD20 antibody.

4. The method of claim 3 wherein the cell culture is a mammalian cell culture.

5. The method of claim 4 wherein the mammalian cells are Chinese Hamster Overy (CHO) cells.

6. The method of claim 4 wherein the purification is performed in the absence of a Protein A purification step.

7. The method of claim 6 wherein the purification is performed in the absence of a cation exchange chromatography step.

8. The method of claim 4 wherein the purification additionally comprises a viral filtration step and an anion exchange chromatography step.

9. The method of claim 8 comprising the steps of (a) crystallizing the CD20 antibody, (b) dissolving the CD20 antibody crystals in a buffer, (c) subjecting the solution obtained from step (b) to anion exchange chromatography, and (d) concentrating the eluate obtained from the anion exchange chromatography.

10. A method of purifying a CD20 antibody from concentrated Harvested Cell Culture Fluid (HCCF) of mammalian cells, comprising the steps of (a) concentrating the HCCF, (b) crystallizing the CD20 antibody, (c) dissolving the CD20 crystals to obtain a CD20 solution, (d) subjecting the CD20 solution to purification on an anion exchange column, and (c) isolating the CD20 antibody.

11. The method of claim 10 wherein the CD20 antibody is a 2H7 antibody.

12. The method of claim 11 wherein the CD20 antibody is selected from the group consisting of 2H7 CD20 antibody variants A-I listed in Table 1.

13. The method of claim 12 wherein the CD20 antibody is selected from the group consisting of 2H7 CD20 antibody variants A, C and H listed in Table 1, having VL and VI-1 pairs of SEQ ID NOs: 1 and 2; SEQ ID NOs: 3 and 4; and SEQ ID NOs: 3 and 5, respectively.

14. The method of claim 11 wherein the HCCF is concentrated to a CD20 antibody concentration at about or greater than 1.5 mg/ml.

15. The method of claim 10 wherein crystallization is performed at a pH of about 6.0 to about 8.0.

16. The method of claim 15 wherein crystallization is performed at a pH of 7.8+/−0.2.

17. The method of claim 10 wherein crystallization is performed at a temperature of about 4° C. to about 40° C.

18. The method of claim 17 wherein crystallization is performed at a temperature of about 37° C.

19. The method of claim 10 wherein crystallization is induced by one or more precipitant selected from the group consisting of PBS, NaCl, Na₂SO₄, KCl, K₂SO₄, Na₂HPO₄, and KH₂PO₄.

20. The method of claim 19 wherein the precipitant is KH₂PO₄.

21. A method of purifying a CD20 antibody from concentrated Harvested Cell Culture Fluid (HCCF) of mammalian cells, comprising the steps of (a) concentrating the HCCF, (b) diafiltering the HCCF with a high salt concentration at a pH that inhibits crystallization, (c) crystallizing the CD20 antibody by raising the pH, (d) dissolving the CD20 antibody crystals to obtain a CD20 antibody solution, (e) subjecting the CD20 antibody solution to purification on an anion exchange column, and (f) isolating the resultant purified CD20 antibody.

22. The method of claim 21 wherein the CD20 antibody is selected from the group consisting of 2H7 CD20 antibody variants A-I listed in Table 1.

23. The method of claim 22 wherein the CD20 antibody is selected from the group consisting of 2H7 CD20 antibody variants A, C and H listed in Table 1, having VL and VH pairs of SEQ ID NOs: 1 and 2; SEQ ID NOs: 3 and 4; and SEQ ID NOs: 3 and 5, respectively.

24. A crystal of a CD20 antibody.

25. The crystal of claim 24 which has a microneedle, needle, globular or globular peanut morphology.

26. A composition comprising a crystal of claim 24.

27. The composition of claim 26 which is a pharmaceutical composition, comprising one or more pharmaceutically acceptable excipients.

28. A method for treating a CD20-associated condition or disease comprising administering to a mammalian subject an effective amount of a CD20 antibody purified by the method of claim 1 or claim 21.
